(19) United States
(12) Patent Application Publication
(10) Pub. No.: US 2008/0262552 A1
(43) Pub. Date: Oct. 23, 2008

(54) Spinal Stabilization Apparatus

(76) Inventor: Soo-Kyung Kim, Seoul (KR)

Correspondence Address: Jason Y. Pahng and Associates, LLC, 12178 Bridgend Run, Fairfax, VA, 22030, US

(21) Appl. No.: 12/094,779

(22) PCT Filed: Oct. 1, 2007
(86) PCT No.: PCT/KR2007/004794

§ 371 Date: May 23, 2008

(30) Foreign Application Priority Data
Dec. 7, 2006 (KR) 10-2006-0124157

(51) Int. Cl. A61B 17/70; A61B 17/04

(52) U.S. Cl. 606/276; 606/246; 606/301

(57) ABSTRACT

A spinal stabilization apparatus can reduce side effects from occurring in free spines adjacent to stiffly stabilized spines and reduce additional injury on a patient by easily extending new rod to adjacent, free spines without removing pre-installed spinal stabilization apparatus during re-surgery. According to one embodiment of the present invention, is provided a spinal stabilization apparatus comprising: at least one first stabilization member which is installed to a first spine; at least one second stabilization member which is installed to a second spine adjacent to the first spine; and a rod comprising one end engaged with the first stabilization member, the other end engaged with the second stabilization member, and a convolution portion formed by rolling the rod at least once in an extension direction from the first spine to the second spine or a curved portion protruding at least once perpendicular to the extension direction.

### TECHNICAL FIELD

The present invention relates to a spinal stabilization apparatus, and more particularly, to a spinal stabilization apparatus, for use in spine surgery, which can be elastically deformed.

### BACKGROUND OF THE INVENTION

In general, spinal diseases, such as disc herniation, spondylolysis, scoliosis, fracture, and instability, require spinal fusion or spinal stabilization together with external nerve decompression when a pathological injury to spinal discs is serious. The spinal stabilization is a surgical technique to reduce the injured spines to a normal status and then stabilize them using a spinal stabilization apparatus.

FIGS. 1A and 1B are a plan view and a perspective view of a conventional spinal stabilization apparatus 30 for use in spinal stabilization, respectively.

Referring to FIGS. 1A and 1B, the conventional spinal stabilization apparatus 30 comprises screws 10 and a rod 20. The screws may be installed by being inserted at a predetermined angle and depth into the pedicle of the spines A and B. The rod 20 is combined with the screw 10 to stabilize the adjacent spines A and B stiffly with each other. The screw 10 may comprise a rod mounting portion 12 which provides a seating groove 13 for the rod 20 so as to combine the rod 20 with an upper portion of the screw 10. The rod mounting portion 12 has a spiral groove 14 formed on an inner circumference thereof. The combining element 15 with a spiral extrusion corresponding to the spiral groove is coupled with the inner circumference, thereby combining the rod 20.

Stabilizing the adjacent spines A and B using the stiff rod 20 is called stiffness stabilization. Generally, the two adjacent spines A and B are stabilized using two screws 10 and one rod 20 so that the movement of joints of the spines A and B is completely suppressed.

The spinal stabilization using the conventional spinal stabilization apparatus 30 is applied primarily to patients who suffer from continuing low-back pain that cannot be eliminated by methods other than surgery. However, in case of the spinal stabilization, it is difficult to predict the results of the surgery. Sometimes, the spinal stabilization may cause worse results than the original status of the patient.

In particular, side effects of the spinal stabilization frequently occur in the free spines A′ and B′ that are directly adjacent to the spines A and B that are stabilized by the conventional spinal stabilization apparatus 30. The side effects produced in the adjacent, free spines A′ and B′ are, for example, disc herniation, degeneration, spinal stenosis, spondylolysis, facet joint arthritis, and instability or the like. These side effects may occur both in the spines A′ and B′ (hereinafter, referred to as topping off and bottom off, respectively) that are adjacent to the head side portion of the stiff stabilized spines A and to the pelvis side portion of the stiff stabilized spines B. It is reported that the above-described side effects may be occurred within 5 to 7 years after the surgery using the conventional spinal stabilizing apparatus 30.

Once the side effects occur in the adjacent free spines A′ and B′, a re-surgery process is generally conducted. The re-surgery process will be conducted by installing a new spinal stabilization apparatus which can cover the topping off and bottom off, after removing the spinal stabilization apparatus 30 installed by the initial surgery. Since the new spinal stabilization apparatus is used in the re-surgery, a patient suffers from additional tissue injury. In addition, since the existing spinal stabilization apparatus 30 must be completely removed before the re-surgery, a long surgery time is required.

### SUMMARY OF THE INVENTION

The present invention provides a spinal stabilization apparatus that can reduce side effects on the free spines adjacent to the spines stiffly stabilized by a stiffness rod, and minimize tissue damage and reduce surgery time by easily being extended to the adjacent, free spines during the re-surgery.

According to an aspect of the present invention, there is provided a spinal stabilization apparatus comprising: at least one first stabilization member which is installed to a first spine; at least one second stabilization member which is installed to a second spine adjacent to the first spine; and a rod comprising one end engaged with the first stabilization member, the other end engaged with the second stabilization member, and a convolution portion formed by rolling the rod at least once in an extension direction from the first spine to the second spine or a curved portion protruding at least once perpendicular to the extension direction.

In some embodiments, the first and second stabilization members may comprise at least one screw installed to a pedicle of the first and second spines, respectively. In other embodiments, the first and second stabilization members may comprise at least one laminar hook which is installed to at least one of a superior laminar and an inferior laminar of the first and second spines, respectively. In another embodiment, the first and second stabilization members may comprise at least one pedicle hook which is installed to at least one of a superior pedicle and an inferior pedicle of the first and second spines, respectively.

According to another aspect of the present invention, there is provided a spinal stabilization apparatus comprising: at least one stabilization member which is installed to a first spine; and a rod comprising one end engaged with the stabilization member, the other end including a hook portion installed to a second spine adjacent to the first spine, and a convolution portion formed by rolling the rod at least once in an extension direction from the first spine to the second spine or a curved portion protruding at least once perpendicular to the extension direction.

The stabilization member may comprise at least one screw installed to a pedicle of the first spine. The stabilization member may comprise at least one laminar hook which is installed to at least one of a superior laminar and an inferior laminar of the first spine. The stabilization member may comprise at least one pedicle hook which is installed to at least one of a superior pedicle and an inferior pedicle of the first spine.

According to yet another aspect of the present invention, there is provided a spinal stabilization apparatus comprising: a plurality of stabilization members which are installed to a pedicle or a laminar of a first spine and are symmetrical with respect to a spinous process of the first spine; and a bridge-shaped rod comprising first and second ends engaged with the stabilization members, respectively, a third end correspondingly surrounding a portion of a spinous process of a second spine adjacent to the first spine, and an elastic portion for stabilizing the first and second spines elastically and which is between the first end and the third end and between the second end and the third end.

In some embodiments, the third end may comprise a seating portion which accommodates at least one of an upper and a lower portion of the spinous process.

The apparatus may comprise further a stabilization unit to facilitate stabilization of the third end and the spinous process. The stabilization unit may comprise a metal thread or carbon fiber.

The spines may have various movements, for example, lateral bending, axial rotation, flexion, and extension by various forces including a weight and a rotational force originated from motions of the human's head, the chest, and the pelvis. If these forces are discontinuously transmitted between the spines stiffly stabilized by the spinal stabilization apparatus and the free spines adjacent thereto, the joint portion of the adjacent, free spines may be damaged.

According to the spinal stabilization apparatus of the present invention, the adjacent spines are elastically stabilized using a rod including a convolution portion or a curved part so that the various forces including compressive, tensile and the rotational force can be prevented from being discontinuously propagated through the spines, and thus, damages on the adjacent free spines can be minimized and prevented.

According to the spinal stabilization apparatus of the present invention, adjacent spines are elastically stabilized using a rod including at least one of convolution portion and a curved part so that a compressive and tensile forces or a rotational force, delivered from free spines adjacent to the elastically-stabilized spines, can be continuously propagated to the elastically-stabilized spines thereby minimizing and prevent the damage of the free spines.

In addition, according to the spinal stabilization apparatus of the present invention, since a pre-installed stabilization apparatus need not to be removed for re-surgery and new spinal stabilization apparatus can extend easily to the adjacent free spines, an internal injury that may be caused additionally due to the re-surgery can be minimized and a surgery time can also be reduced.

### BRIEF DESCRIPTION OF THE DRAWINGS

FIGS. 1A and 1B are a plan view and a perspective view of a conventional spinal stabilization apparatus for use in spinal stabilization, respectively.

FIGS. 2A through 3D are perspective views of various spinal stabilization apparatuses according to embodiments of the present invention, respectively.

FIGS. 4A through 4D are perspective views illustrating a method of applying the spinal stabilization apparatuses shown in FIGS. 2A through 3D, respectively.

FIGS. 5A through 5C are perspective views of spinal stabilization apparatuses according to other embodiments of the present invention, respectively.

FIGS. 6A and 6B are a plan view and a perspective view of a spinal stabilization apparatus according to another embodiment of the present invention, respectively.

FIGS. 7A and 7B are a plan view and a perspective view of a spinal stabilization apparatus according to another embodiment of the present invention, respectively.

FIG. 8 is a perspective view of a spinal stabilization apparatus according to another embodiment of the present invention.

FIGS. 9A and 9B are perspective views of a spinal stabilization apparatus according to another embodiment of the present invention, respectively.

FIG. 10 is a perspective view of a spinal stabilization apparatus according to another embodiment of the present invention.

FIG. 11 is a perspective view of a spinal stabilization apparatus according to another embodiment of the present invention.

FIGS. 12A and 12B are a plan view and a perspective view of a spinal stabilization apparatus according to another embodiment of the present invention, respectively.

FIGS. 13A and 13b are a plan view and a perspective view of a spinal stabilization apparatus according to another embodiment of the present invention, respectively.

FIGS. 14A and 14B are a plan view and a perspective view of a spinal stabilization apparatus according to another embodiment of the present invention, respectively.

FIGS. 15A and 15B are a top view and a perspective view of a spinal stabilization apparatus according to another embodiment of the present invention, respectively.

FIGS. 16A and 16B are perspective views of a spinal stabilization apparatus according to another embodiment of the present invention, respectively.

### DETAILED DESCRIPTION OF THE BEST MODE

The present invention will now be described more fully with reference to the accompanying drawings, in which exemplary embodiments of the invention are shown. The present invention may, however, be embodied in many different forms and should not be construed as being limited to the embodiments set forth herein; rather, these embodiments are provided so that this disclosure will be thorough and complete, and will fully convey the concept of the invention to one skilled in the art.

In the drawings, the thicknesses of layers and regions are exaggerated for clarity. Like reference numerals in the drawings denote like elements, and thus their description will be omitted. As used in the present specification, the terms “and/or” include any one of listed items or all combinations of one or more items. In the present specification, the terms such as “first” and “second” are used to explain various members, components, regions, layers and/or portions. However, it is self-obvious that these members, components, regions, layers and/or portions should not be construed as being limited to the terms. These terms are used only to discriminate one member, component, region, layer or portion from other region, layer or portion. Thus, a first member, component, region, layer or portion that will be described as below may denote a second member, component, region, layer or portion without escaping from the teaching of the invention.

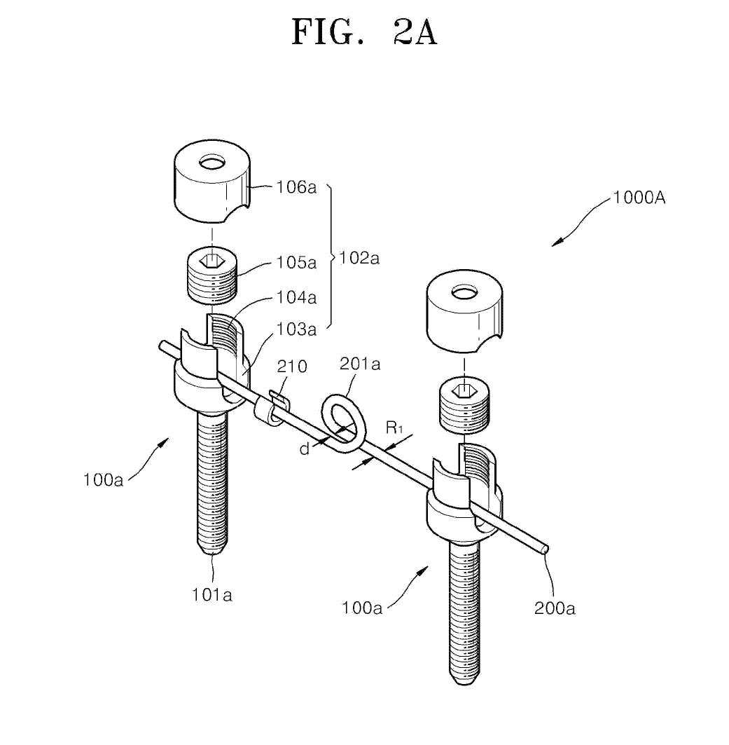

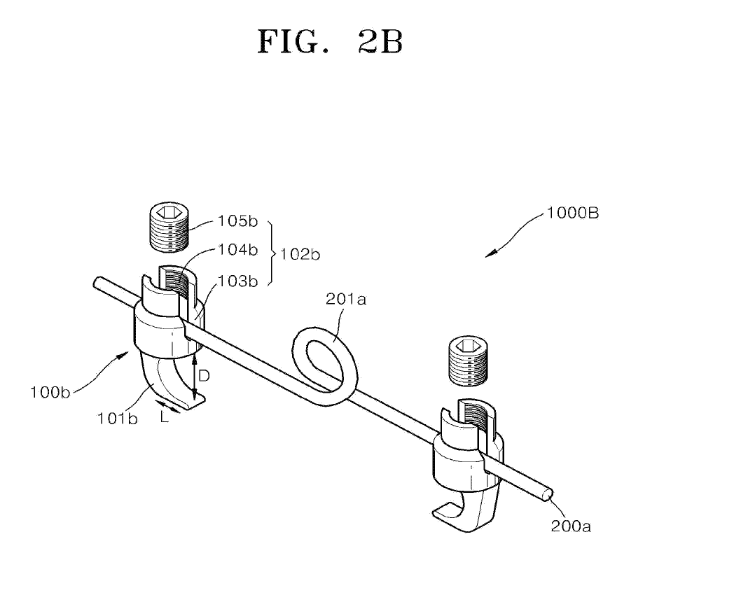

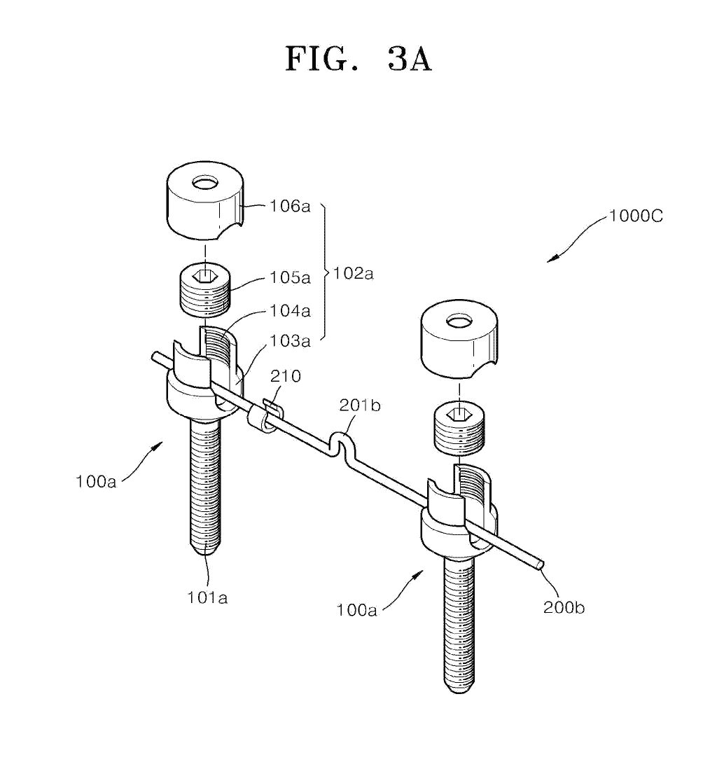

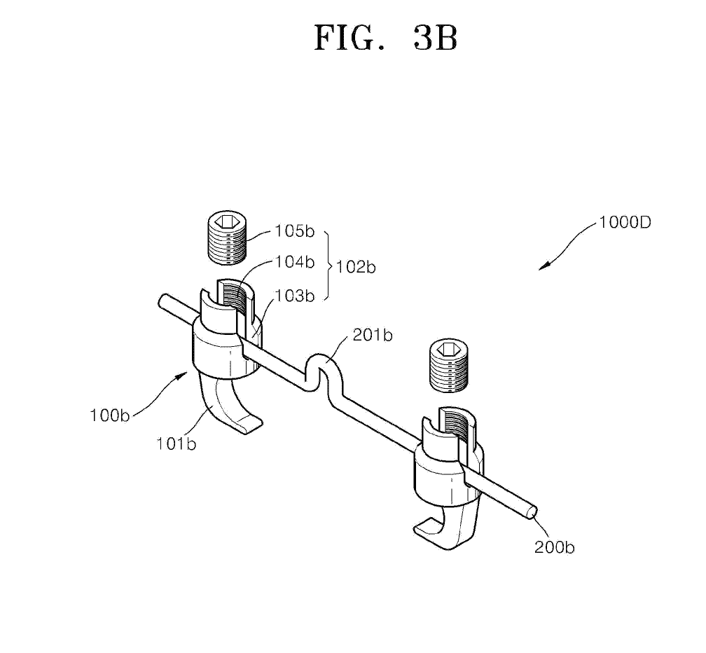

FIGS. 2A through 3D are perspective views of various spinal stabilization apparatuses 1000A, 1000B, 1000C, 1000D, 1000E, and 1000F according to various embodiments of the present invention, respectively.

Referring to FIG. 2A, the spinal stabilization apparatus 1000A comprises a stabilization member 100a that can be installed by being inserted into a pedicle of the spines. The stabilization member 100a may be a screw with a body portion 101a on the surface of which a helix is formed. Referring to FIG. 2B, the spinal stabilization apparatus 1000B comprises at least one of a laminar hook and a pedicle hook as a stabilization member 100b. The laminar hook and pedicle hook include a body portion 101b with a hook to grab and be fixed to a laminar or a pedicle of the spines, respectively. The body portion 101b have different widths D and lengths L 101b depending on the place of the spine with the body portion 101b is to be combined, while the shapes of the laminar hook and pedicle hook may be generally similar to each other.

Each of the stabilization members 100a and 100b comprises rod mounting portions 102a and 102b which provide seating grooves 103a and 103b which the rod 200a is seated on and passes through. Each of the rod mounting portions 102a and 102b may be engaged with engaging members 105a and 105b having a helix corresponding to a helix formed on inner circumferences 104a and 104b of the seating grooves 103a and 103b, so as to couple the rod 200a with the stabilization members 100a and 100b. Each of the stabilization members 100a and 100b may further comprise a cover 106a, as shown in FIG. 2A, so as to maintain the state in which the rod mounting portions 102a and 102b, the rod 200a and the engaging member 105a are engaged with one another. In other embodiments of the present invention, the rod mounting portions 102a and 102b may comprise a hole-shaped seating portion through which the rod 200a can pass through.

The spinal stabilization apparatuses 1000A and 1000B comprise the rod 200a of which both ends are respectively engaged with the rod mounting portions 102a and 102b of the stabilization members 100a and 100b. The rod 200a may comprise a convolution portion 201a that stabilizes the adjacent spines elastically.

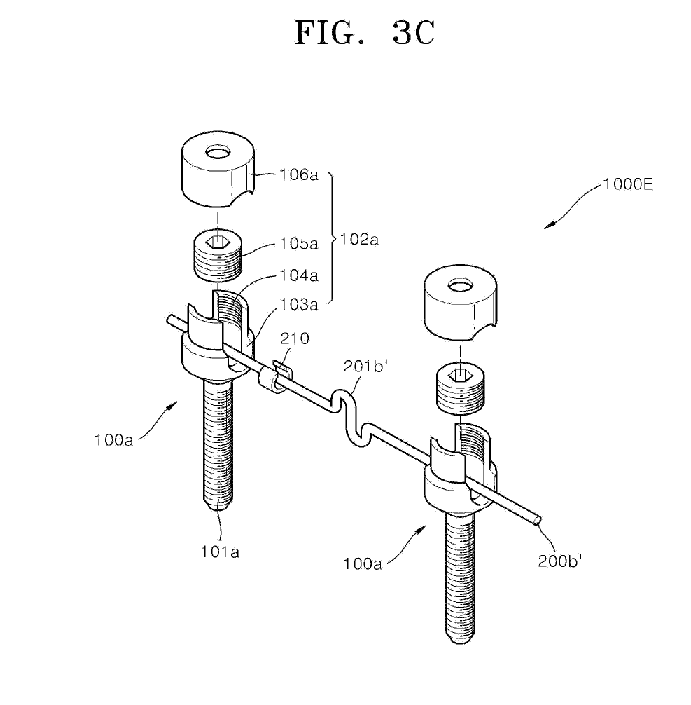

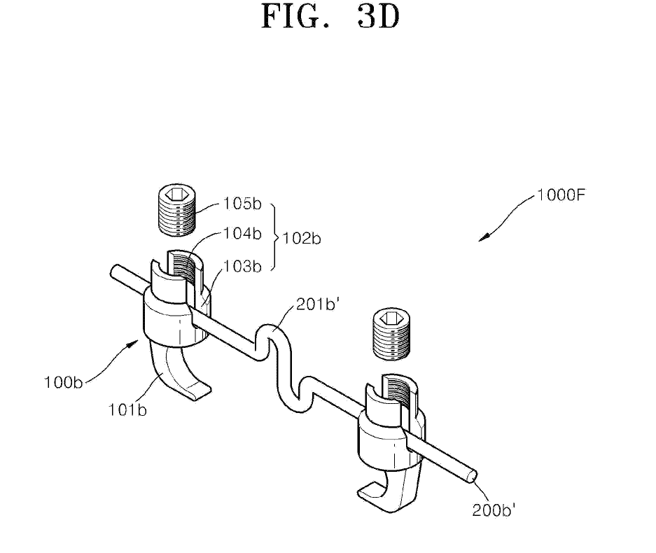

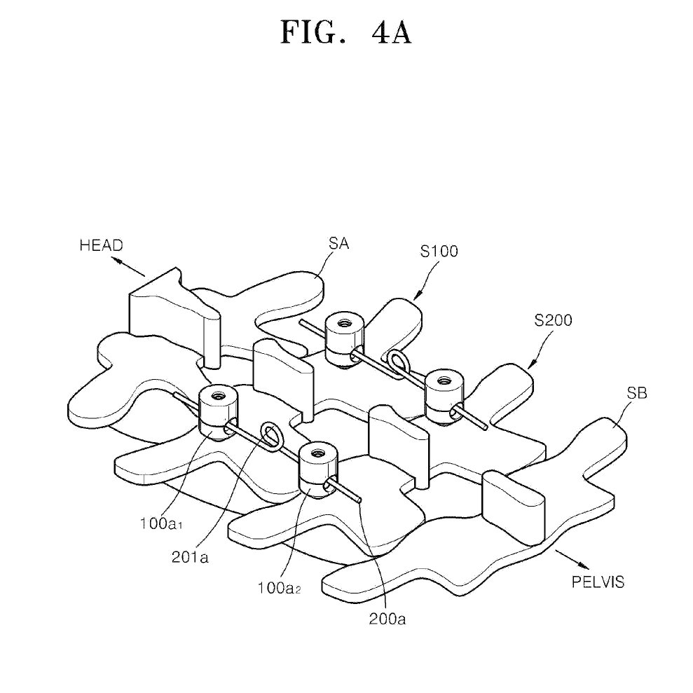

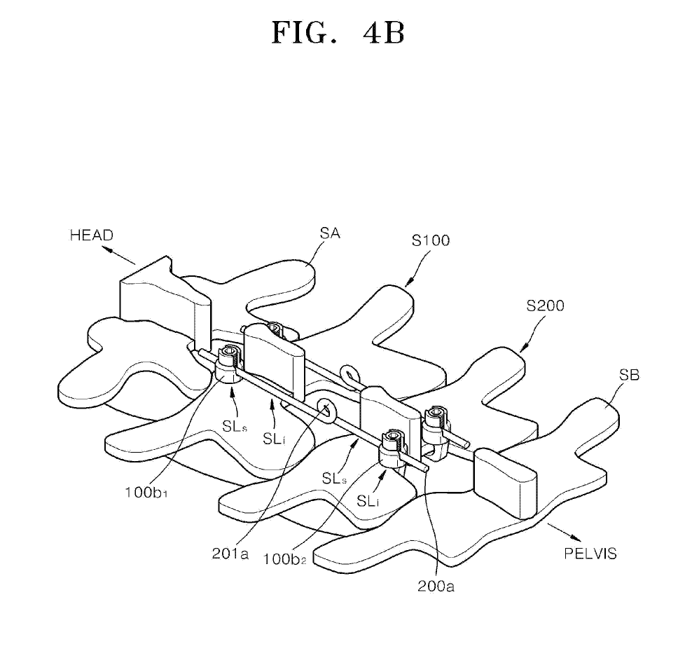

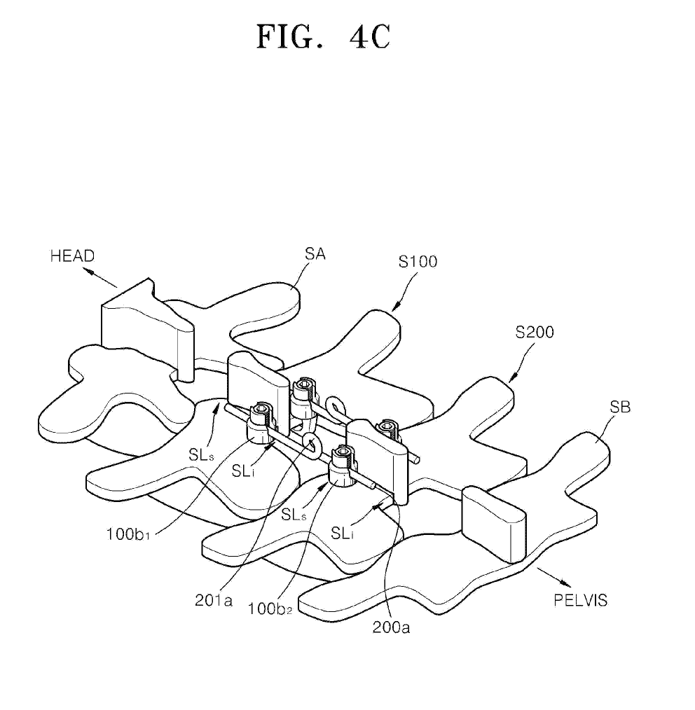

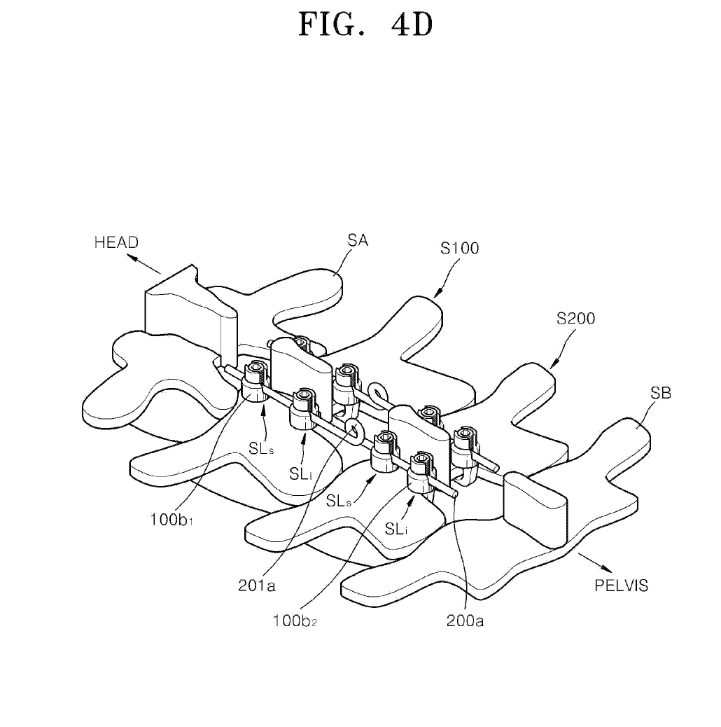

The convolution portion 201a of the rod 200a may be formed by rolling a straight rod into a loop formation in an extending direction of a first spine S100 to a second spine S200 (refer to FIGS. 4A and 4B). The convolution portion 201a may be rolled twice or more so as to provide elasticity as required by patients. The convolution portion 201a may be rolled to have a distance d at the bottom of the convolution portion 201a in the range of 0.5-2 mm in order for the convolution portion 201a not to contact itself at the bottom thereof. If the convolution portion 201a contacts itself at the bottom of the loop, friction occurs in the convolution portion 201a and contamination may occur in the patient's body.

The rod 200a may be formed of harmless metal such as titanium or an alloy thereof. The cross-section of the rod 200a may be circular or polygonal so as to facilitate bending of the rod 200a in a predetermined direction or so as to prevent the rod 200a from being bent in a predetermined direction.

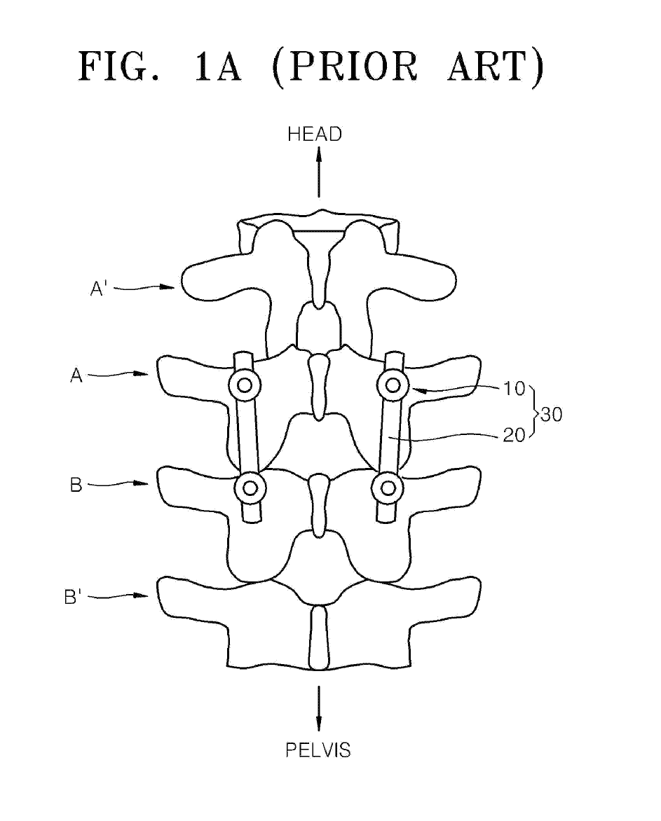

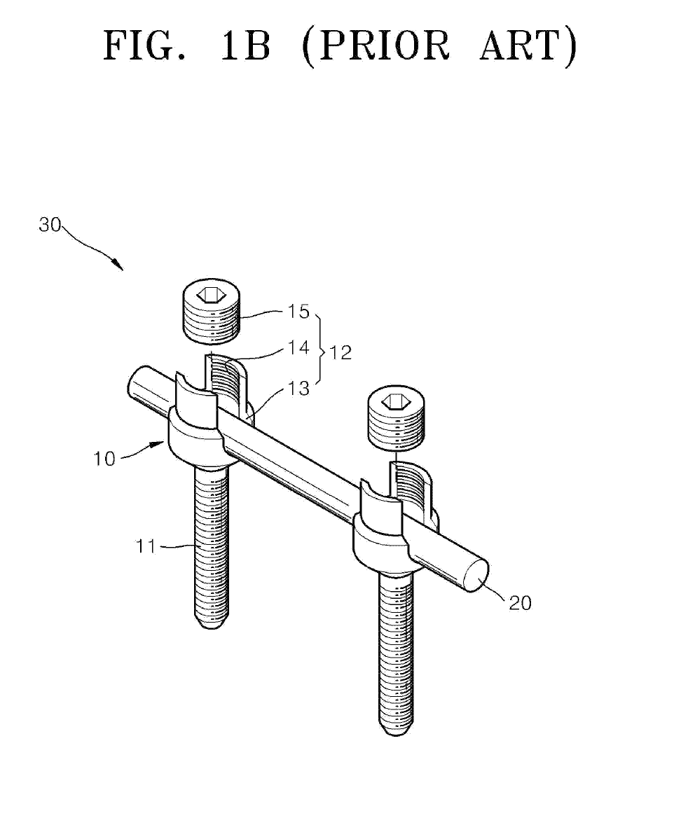

The diameter R₁ of the rod 200a may be 2-5 mm, for example. There is sometimes a case that among a stiff rod 20 and screws 10 (refer to FIG. 1A) which are previously installed by initial surgery, the stiff rod 20 is only removed and the screws 10 are reutilized for new re-surgery of a patient. In general, the commercially available rod 20, which is stiff, has a diameter of 4-7 mm. Thus, a seating groove 13 of the screw 10, for engaging the rod 20 may be larger than the diameter R1 of the rod 200a according to the present embodiment of the present invention. In this case, a compensation member 210, which surrounds the outer circumference of the rod 200a, may be used to compensate for the smaller diameter R1 of the rod 200a. As a result, the rod 200a can replace the pre-installed stiff rod 20 without removing the screw 10 so that additional internal injury to the patient caused by spinal stabilization can be minimized.

The compensation member 210 may include a ring of which a portion is open, as shown in FIG. 2A. The ring-shaped compensation member 210 allows the rod 200a to be easily inserted therein. When the compensation member 210 is engaged with rod mounting portions 102a and 102b, the inner diameter of the compensation member 210 is reduced by a pressing force of the engaging member 105a and the rod 200a may be pressurized and tightened in the seating groove 103a.

Referring to FIGS. 3A through 3D, the spinal stabilization apparatuses 1000C, 1000D, 1000E, and 1000F, according to other embodiments of the present invention, comprise the stabilization members 100a and 100b as described above with reference to FIGS. 2A and 2B. In addition, the spinal stabilization apparatuses 1000C, 1000D, 1000E, and 1000F comprise rods 200b and 200b′ of which both ends are engaged with rod mounting portions 102a and 102b of the stabilization members 100a and 100b, respectively.

In the present embodiments, the rods 200b and 200b′ comprise curved portion 201b and 201b′ which protrude at least once perpendicularly with respect to an extension direction of the rods 200b and 200b′ from first spine S100 to the second spine S200. The curved configurations 201b and 201b′ may protrude once or more so as to provide elasticity as required by patients.

The diameter of each of the rods 200b and 200b′ may be 2-5 mm, for example. In addition, the rod 200b or 200b′ may be formed of harmless metal, such as titanium or an alloy thereof. The cross-section of the rod 200b or 200b′ may be circular or polygonal so as to facilitate bending of the rod 200b or 200b′ in a predetermined direction or so as to prevent the rod 200b or 200b′ from being bent in a predetermined direction. In addition, a compensation member 210 which surrounds the outer circumference of the rod 200b or 200b′ may be used, so as to continue use of the conventional stabilization member 10 pre-installed for a previous initial surgery, as described above.

FIGS. 4A through 4D are perspective views illustrating a method of applying the spinal stabilization apparatuses 1000A, 1000B shown in FIGS. 2A and 2B, respectively to a spines S100 and S200 of patient. However, it should be understood that FIGS. 4A through 4D can also illustrate a method of applying the spinal stabilization apparatuses 1000C-1000F shown in FIGS. 3A through 3D, respectively to a spines S100 and S200 of patient.

Referring to FIG. 4A, a first stabilization member 101a₁ and a second stabilization member 100a₂, which are screw type stabilization members may be installed by being inserted into a pedicle of the first spine S100 and the second spine S200 that are adjacent to each other, respectively. The rod 200a of which both ends are engaged with the first stabilization member 101a₁ and the second stabilization member 100a₂ stabilizes the first spine S100 and the second spine S200 and may provide adjustable elasticity to the first spine S100 and the second spine S200 by the convolution portion 201a of the rod 200a. As a result, a compressive or tensile force and a rotative force delivered from free spines SA and SB that are adjacent to the first spine S100 and the second spine S200, respectively, are continuously propagated through the first spine S100 and the second spine S200 and, accordingly, do not damage the adjacent free spines SA and SB. FIG. 4A illustrates the rod 200a including the convolution portion 201a, however the rod 200b including the curved portion 201b may also be applied to patients in the same manner.

Referring to FIGS. 4B through 4D, a laminar hook may be used as stabilization members 100b₁ and 100b₂ to stabilize at least one of a superior laminar SLs and an inferior laminar SLi of the first spine S100 with at least one of an inferior laminar SLs and an inferior laminar SLi of the second spine S200. The rod 200a including the convolution portion 201a may minimize damage on the adjacent, free spines SA and SB.

FIGS. 4B through 4D illustrate the rod 200a including the convolution portion 201a, however the rod 200b including the curved portion 201b may also be applied to patients in the same manner. In addition, although not shown in FIGS. 4B through 4D, a pedicle hook may be also be applied as the stabilization members 100b1 and 100b2, instead of the laminar hook, to stabilize the first and second spines S100 and S200.

The rods 200a and 200b according to the present embodiment stabilize the adjacent spines S100 and S200 elastically by the convolution portion 201a or the curved portion 201b and 201b′ of rods 200a and 200b, thereby preventing a compressive or tensile force and a rotational force generated from motions of the head, the chest, and the pelvis of a patient from being discontinuously propagated between the free spines SA and SB and the spines S100 and S200 that are elastically-stabilized by the spinal stabilization apparatus according to the present invention. In particular, the convolution portion 201a and the curved portion 201b and 201b′ according to the present embodiment are adjustably bent and extend in a predetermined direction, for example, a spinal direction so that movements such as lateral bending, axial rotation, flexion, and extension of the elastically-stabilized spines S100 and S200 are available and the spines can be stabilized. As a result, the spinal stabilization apparatus according to the present invention allows the stabilized spines S100 and S200 and the unstabilized spines SA and SB not to be adjacent discontinuously to each other so that side effects on the unstabilized adjacent spines SA and SB as well as the elastically-stabilized spines S100 and S200 can be reduced.

In addition, even when side effects occur in adjacent spines of a patient who experienced a surgery of spinal stabilization, a rod according to the present invention, in the place of a pre-installed conventional rod can be engaged with a stabilization member without removing a screw of a conventional spinal stabilization apparatus so that re-surgery can be easily conducted. As a result, according to the present embodiment, additional internal injury caused by re-surgery may be reduced from occurring.

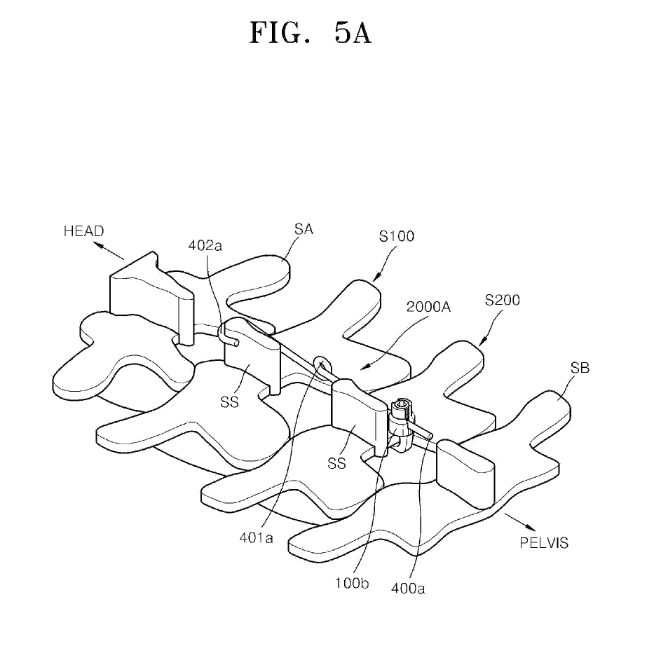

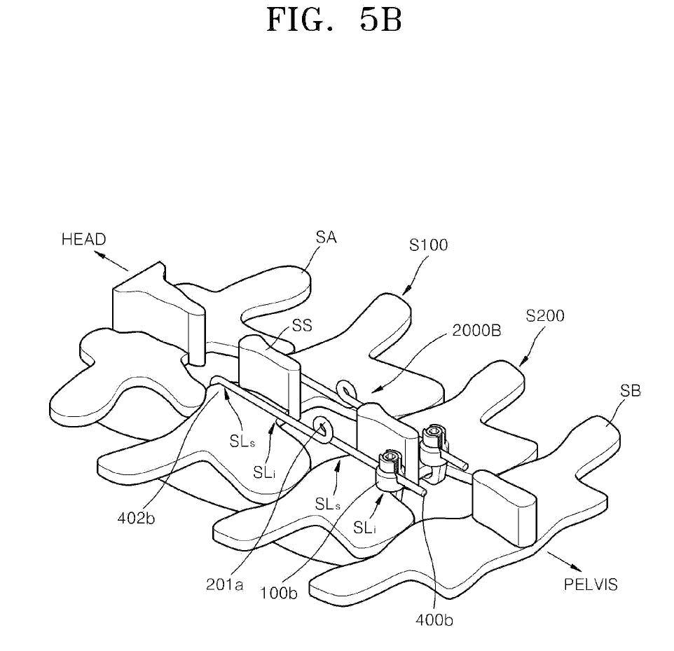

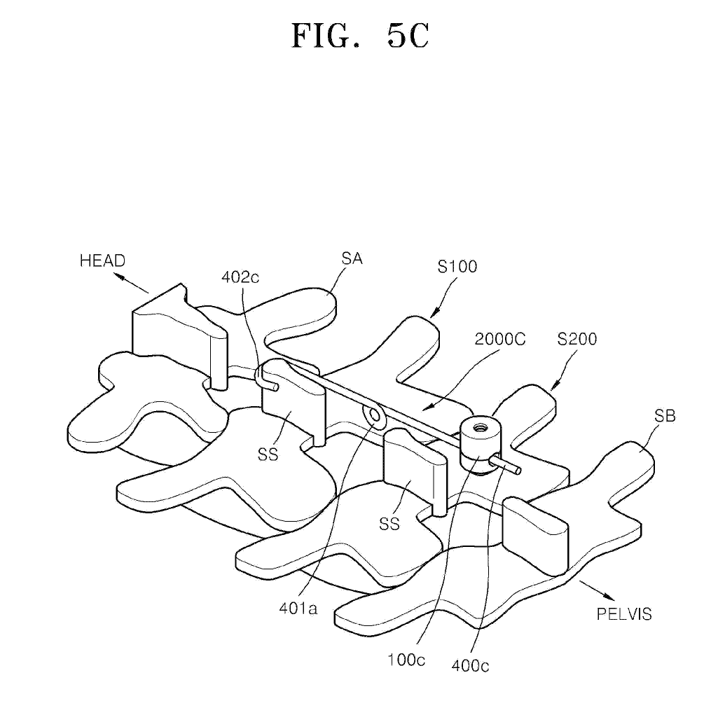

FIGS. 5A through 5C are perspective views of spinal stabilization apparatuses 2000A, 2000B, and 2000C, according to other embodiments of the present invention, respectively.

Referring to FIGS. 5A through 5C, the spinal stabilization apparatuses 2000A and 2000B according to the present embodiments comprise a laminar hook-shaped stabilization member 100b installed to the second spine S200. Selectively, the spinal stabilization apparatuses 2000A may comprise a pedicle hook-shaped stabilization member as a stabilization member. In addition, the spinal stabilization apparatus 2000C may comprise a screw-shaped stabilization member 100c as a stabilization member, as illustrated in FIG. 5C. One end of rods 400a, 400b, and 400c are fixed to the second spine S200 by engaging with the stabilization members 100b and 100c installed on the second spine S200, and the other ends of the rods 400a, 400b, and 400c are fixed to the first spine S100 by hook portions 402a, 402b, and 402c. The rods 400a, 400b, and 400c may be detachably installed to the first spine S100 by the hook portions 402a, 402b, and 402c of the rods 400a, 400b, and 400c without an additional stabilization member for the first spine S100. The hook portions 402a, 402b and 402c may be hung and fixed to a spinous process SS of the first spine S100, as illustrated in FIGS. 5A and 5C, or be hung and fixed to the superior laminar SLs of the first spine S100, as illustrated in FIG. 5B. Although not shown, like the spinal stabilization apparatus 2000C of FIG. 5C, the rod 400c stabilized in the screw-shaped stabilization member 100c may be stabilized by being hung in superior laminar SLₛ of the first spine S100.

In FIGS. 5A through 5C, the hook portions 402a, 402b, and 402c are directed toward the head. However, the hook portions 402a, 402b, and 402c may be fixed to a spinous process SS toward a pelvis or an inferior laminar SLi of the spine. In addition, lamina hook type stabilization member 100b can be fixed to the superior laminar SLs of the second spine S200 and the inferior laminar SLi of the first spine S100 while the laminar hook-shaped stabilization member 100b is illustrated to be fixed to the lower laminar SLi of the second spine S200 in FIGS. 5A and 5B. In order to stabilize the first spine S100 and the second spine S200 elastically, the rods 400a and 400b may comprise convolution portions 401a and 401b, as illustrated in FIGS. 5A through 5C, or curved portions 201b and 201b′, as illustrated in FIGS. 3A through 3D.

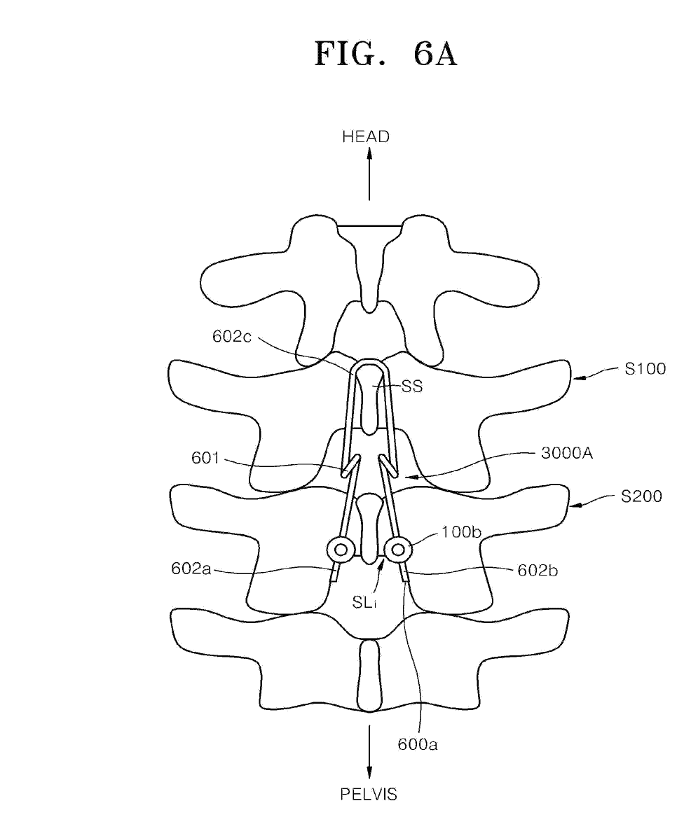

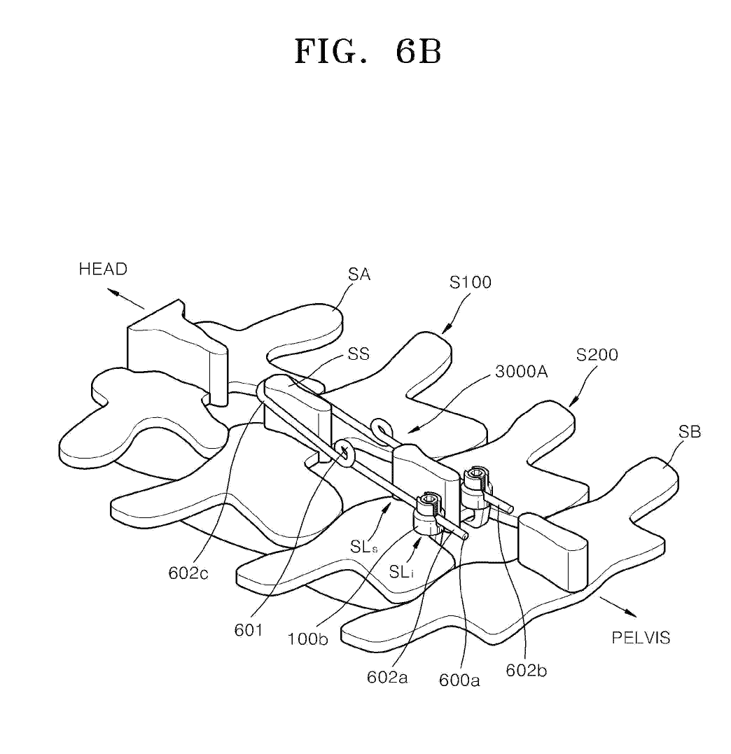

FIG. 6A is a plan view of a spinal stabilization apparatus 3000A according to another embodiment of the present invention, and FIG. 6B is a perspective view of the spinal stabilization apparatus 3000A illustrated in FIG. 6A.

Referring to FIGS. 6A and 6B, the spinal stabilization apparatus 3000A comprises a laminar hook-shaped stabilization member 100b and a bridge-shaped rod 600a. The laminar hook-shaped stabilization member 100b is fixed symmetrically to a laminar of the second spine S200 to be centered at a spinous process of second spine S200. A first end 602a and a second end 602b of the bridge-shaped rod 600a are engaged with the laminar hook-shaped stabilization member 100b, respectively.

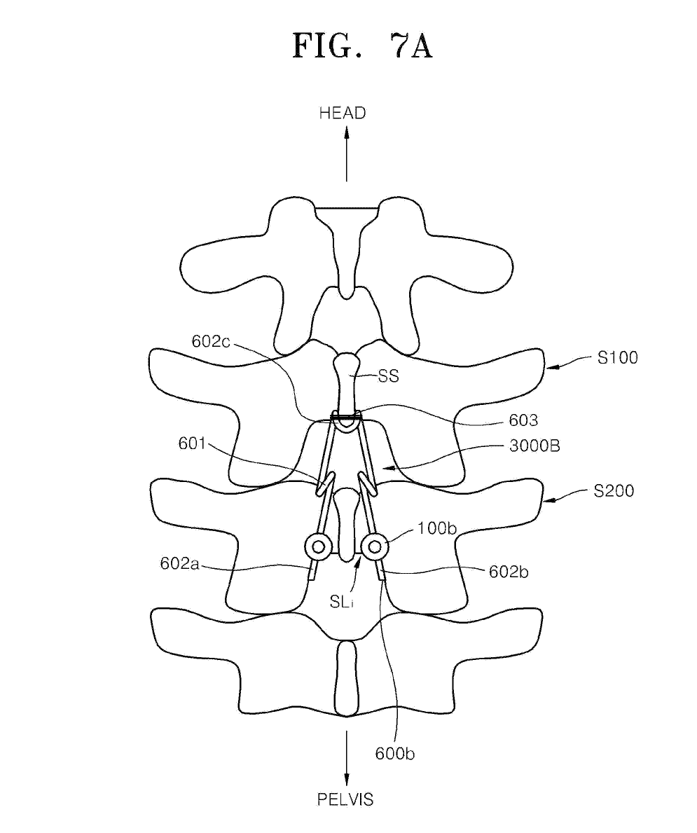

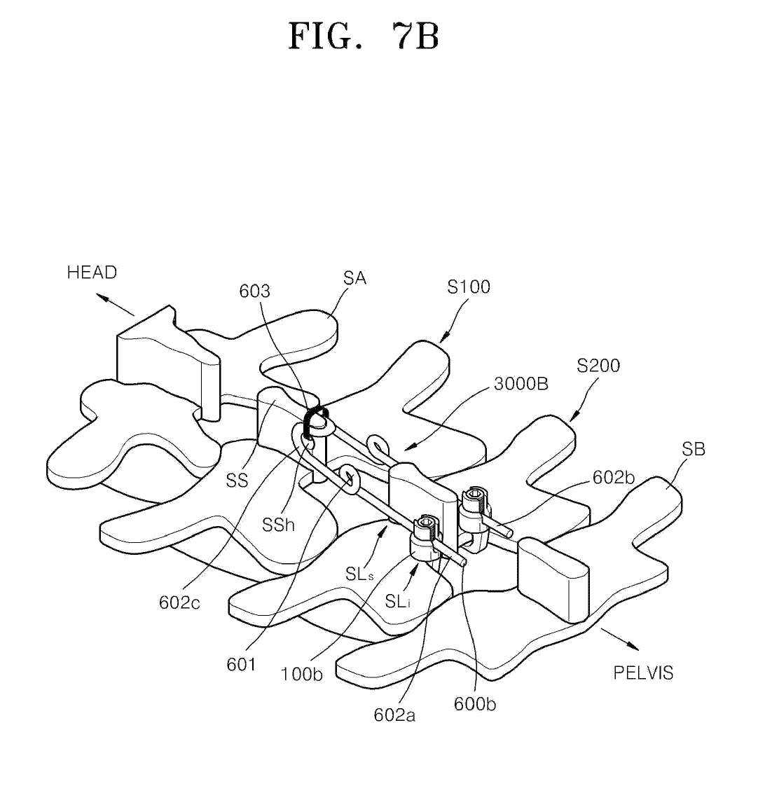

In the present embodiment of the present invention, a third end 602c of the rod 600a is fixed to a spinous process SS of the first spine S100 with the third end 602C surrounding a portion of the spinous process SS, for example, a side portion of the spinous process SS toward a head, upper portion of the spinous process SS, as illustrated in FIGS. 6A and 6B. The third end 602c may be shaped into a prop to accommodate the upper portion of the spinous process SS stably, similar as will be illustrated in FIG. 7A.

The rod 600a comprises a convolution portion 601 between the first end 602a and the third end 602c and between the second end 602b and the third end 602c, respectively, to help stabilize elastically the spines S100 and S200 and prevent damage to the adjacent first and second spines S100 and S200.

FIG. 7A is a plan view of a spinal stabilization apparatus 3000B according to another embodiment of the present invention, and FIG. 7B is a perspective view of the spinal stabilization apparatus 3000B illustrated in FIG. 7A.

Referring to FIGS. 7A and 7B, a third end 602c is formed corresponding to a portion of the spinous process SS toward a pelvis, i.e. a bottom portion of the spinous process SS of the first spine S100, unlike the third end 602a of FIG. 6A. In addition, the third end 602c may be formed into a prop to accommodate the bottom portion of the spinous process SS of the first spine S100. The structure of the prop can be provided by folding the rod 600b at least once. The bridge-shaped rod 600b may have the convolution portion 601 so as to stabilize the first spine S100 and the second spine S200 elastically.

In some embodiment of the present invention, as shown in FIG. 7A, a stabilization unit 603, for fixing the third end 602c with the portion of the spinous process SS of the first spine S100, may be used. To provide the stabilization unit 603, a through-hole SSh is formed in the spinous process SS and engaging units such as bolt and nut are inserted and coupled with each other, thereby facilitating the fixation of the third end 602c to the spinous process SS. In other embodiments of the present invention, the third end 602c may be fixed to the spinous process SS by using a harmless metal thread or a wire or carbon fiber as the stabilization unit 603.

Since the bridge-shaped rods 600a and 600b according to the present embodiment help stabilize the adjacent spines S100 and S200 symmetrically with respect to the spinous process SS of the spine S 200, the force delivered from the free spines SA and SB can be propagated symmetrically.

In FIGS. 6A and 7B, the third end 602c is directed toward the head of a patient. However, the third end 602c may be directed toward the pelvis of a patient. In this case, the third end 602c may be fixed to an upper or bottom portion of the spinous process SS of the spine S200. In addition, the laminar hook-shaped stabilization member 100b is not only fixed to an inferior laminar SLi of the second spine S200, however may also be fixed to superior laminar SLs of the second spine S200 or the inferior laminar SLi of the first spine S100. It is obvious to one skilled in the art that a screw-shaped stabilization may also be used as a stabilization member. In order to stabilize the first spine S100 and the second spine S200 elastically, the rods 600a and 600b may comprise the curved portions 201b and 201b′ as a elastic portion, as shown in FIGS. 3A through 3D.

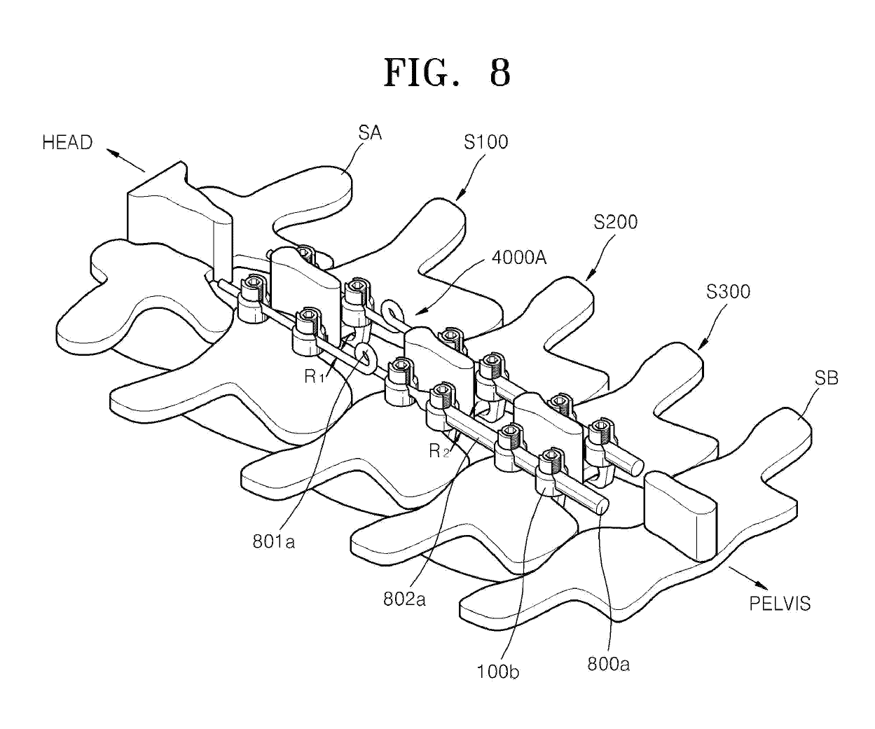

FIG. 8 is a perspective view of a spinal stabilization apparatus 4000A according to another embodiment of the present invention.

Referring to FIG. 8, the spinal stabilization apparatus 4000A comprises laminar hook-shaped stabilization members 100b that are installed to three adjacent spines S100, S200, and S300, respectively. Rods 800a, that engage with the stabilization members 100b, comprise an elastic portion 801a which stabilizes the first spine S100 and the second spine S200 elastically and a stiffness portion 802a which extends in the direction of the third spine S300 from the elastic portion 801a and stabilizes the second spine S200 and the third spine S300 stiffly.

The elastic portion 801a may include a convolution portion 201a or the curved portions 201b and 201b′ as shown in FIGS. 3A through 3D. The elastic portion 801a and the stiffness portion 802a may be formed as one body, as shown in FIG. 8, and the diameter R2 of the stiffness portion 802a may be larger than the diameter R1 of the elastic portion 801a. For example, the diameter R2 of the stiffness portion 802a may be 4-7 mm, and the diameter of the elastic portion 801a may be 2-5 mm.

FIG. 8 shows the rod 800a having one elastic portion 801a and one stiffness portion 802a. However, the rod 800a may further comprise another stiffness portion extending from the elastic portion 801a to another spine SA or another elastic portion extending from the stiffness portion 802a to another spine SB. In this case, a stabilization member such as a screw, a laminar hook or a pedicle hook can be further installed to the spine SA and/or the spine SB to engage these further extended portions.

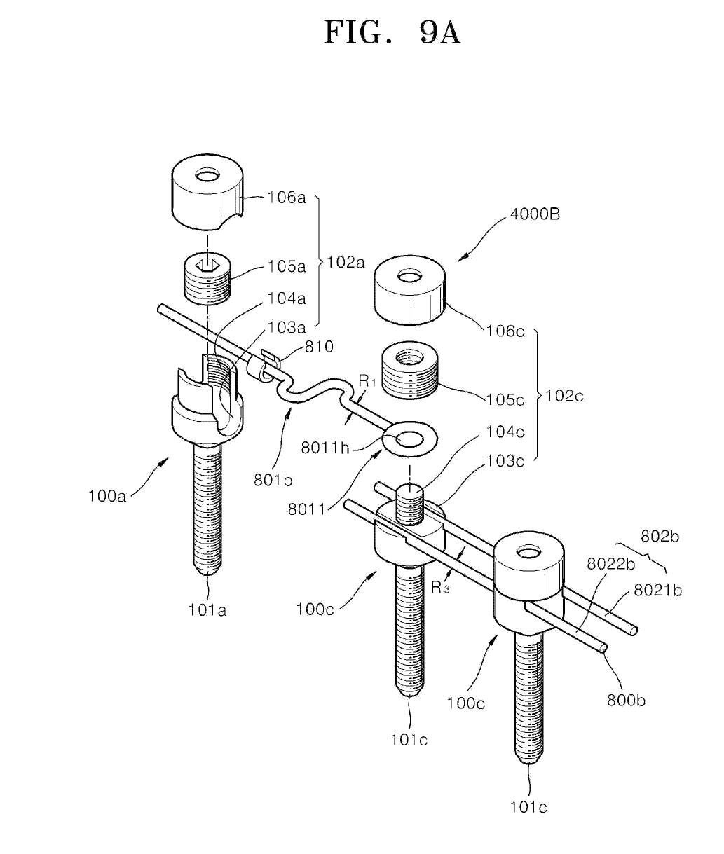

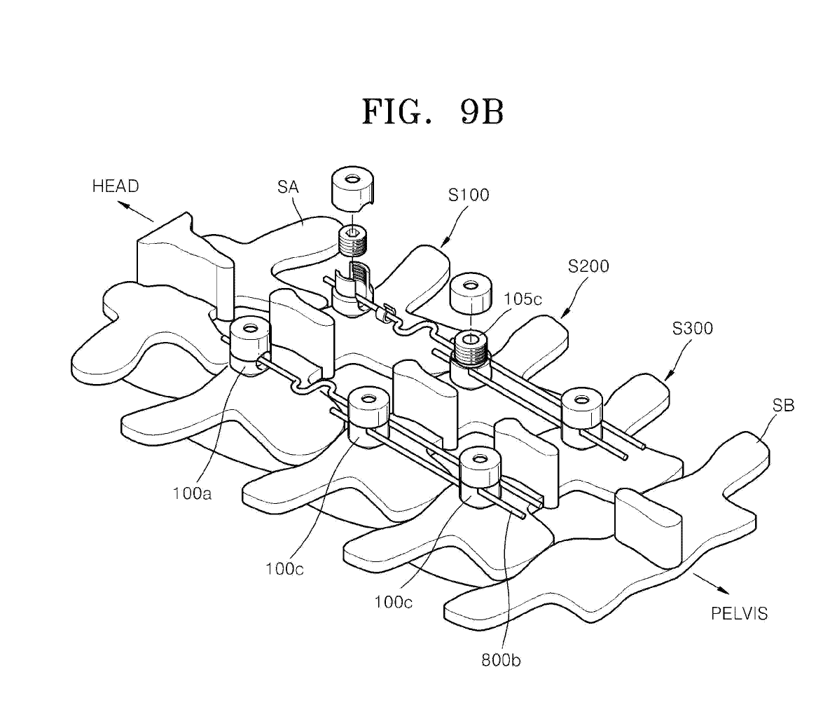

FIGS. 9A and 9B are perspective views of a spinal stabilization apparatus 4000B according to another embodiment of the present invention, respectively.

Referring to FIGS. 9A and 9B, the spinal stabilization apparatus 4000B according to the present embodiment comprises a rod 800b that is non-integrally formed of an elastic portion 801b and a stiffness portion 802b, unlike the rod 800a of FIG. 8. The elastic portion 801b may include a curved portion 201a or a convolution portion 201b and 201b′, as shown in FIGS. 2A to 3D.

The stiffness portion 802b may comprise two subrods 8021b and 8022b. For example, the diameter R1 of the elastic portion 801b and the diameter R3 of each the subrods 8021b and 8022b of the stiffness portion 802b may be 2-5 mm, and the diameter R1 of the elastic portion 801b and the diameter R3 of each of the subrods 8021b and 8022b of the stiffness portion 802b may be substantially equivalent.

The rod 800b may be engaged with the screw-shaped stabilization members 100a and 100c which are installed to a pedicle of the spines S100, S200, and S300, and thus, stabilize the spines S100, S200, and S300. When the elastic portion 801b and the stiffness portion 802b are non-integrally formed, one end of the elastic portion 801b and one end of the stiffness portion 802b may be commonly engaged with the stabilization member 100c.

A rod mounting portion 102c of the stabilization member 100c, for commonly engaging the elastic portion 801b and the stiffness portion 802b, comprises grooves 103c on which the subrods 8021b and 8022b of the stiffness portion 802c are seated, a protrusion 104c having a helix formed and protruding between the grooves 103c, and an engaging member 105c which is able to screw-engage with the protrusion 104c.

One end of the elastic portion 801b may comprise a disk 8011 with a through-hole 8011h which is formed to be able to be detachably installed to the protrusion 104c. After the subrods 8021b and 8022b of the stiffness rod 802c are first seated in the grooves 103c, the disk 8011 is disposed on the subrods 8021b and 8022b and the engaging member 105c is engaged with protrusion 104c, so that the disk 8011 and the subrods 8021b and 8022b are compressed together and may be fixed with the rod mounting portion 102c. The rod mounting portion 102c may further comprise a cover 106c, so as to further maintain the engaged status between the protrusion 104c and the engaging member 105c.

FIGS. 9A and 9B illustrate the screw-shaped stabilization members 100a and 100c. However, it is obvious to one skilled in the art that the laminar hook or pedicle hook-shaped stabilization member 100b as shown in FIG. 2B may be used as a stabilization member. In addition, a stiffness portion does not have to be the subrods 8021b and 8022b. The stiffness portion can also be a rod 20 formed of a single body, as shown in FIG. 1B and It is obvious from this disclosure that an end of the rod 20 can be modified in order to commonly engage the rod and the elastic portion 801b using the stabilization member 100c. For example, the end of the rod 20 can be formed into a disk with a through hole. In addition, it should be understood that a spinal stabilization apparatus may further comprise another elastic portion extending from the stiffness portion 802 and a stabilization member to engage the elastic portion.

FIG. 10 is a perspective view of a spinal stabilization apparatus 4000C according to another embodiment of the present invention.

Referring to FIG. 10, the spinal stabilization apparatus 4000C comprises screws 250 which are installed to at least two adjacent spines S200 and S300, a first rod 260a and 260b which is engaged with the screws 250, respectively to connect the screws 250 stiffly in a line, and a second rod 282 which is engaged with the screw 250 installed to an adjacent spine S100 to connect the screw 250 and the first rod elastically. The spinal stabilization apparatus 4000C has a structure in which the first rods 260a and 260b and the second rod 282 are non-integrally or separately formed, like the rod 800b of FIGS. 9A and 9B.

The screw 250 as shown in FIG. 2A may be used as a stabilization member. The screw 250 is installed to a pedicle or a sacrum of the spine S100, S200, and S300, and the rods 260a, 260b or 282 is engaged with the upper portions of the screw 250.

The first rods 260a and 260b connect the screws 250A and 250B stiffly in a line. A laminar hook or a pedicle hook instead of a screw may be used, as illustrated in FIG. 10, as the screw 250.

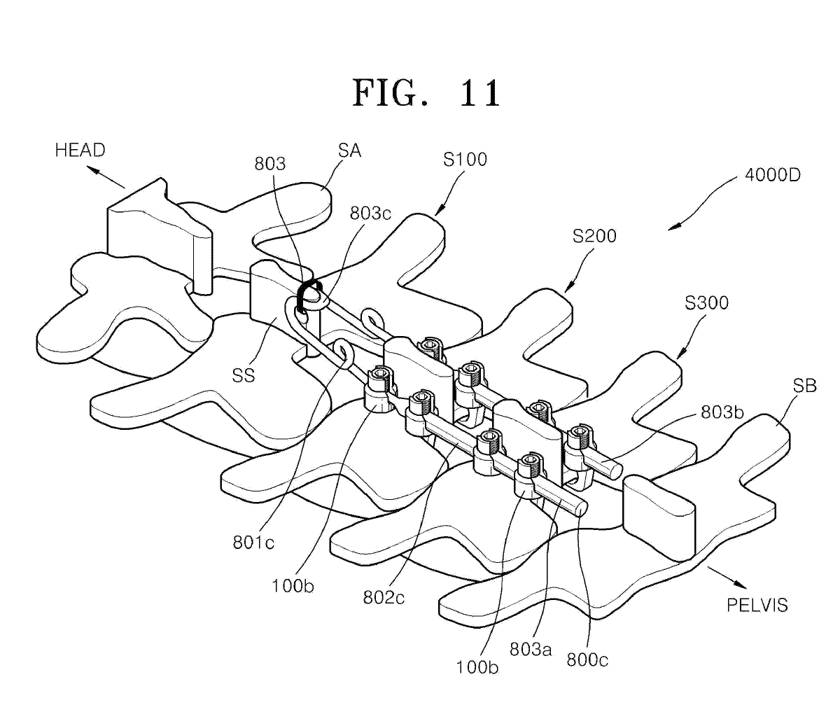

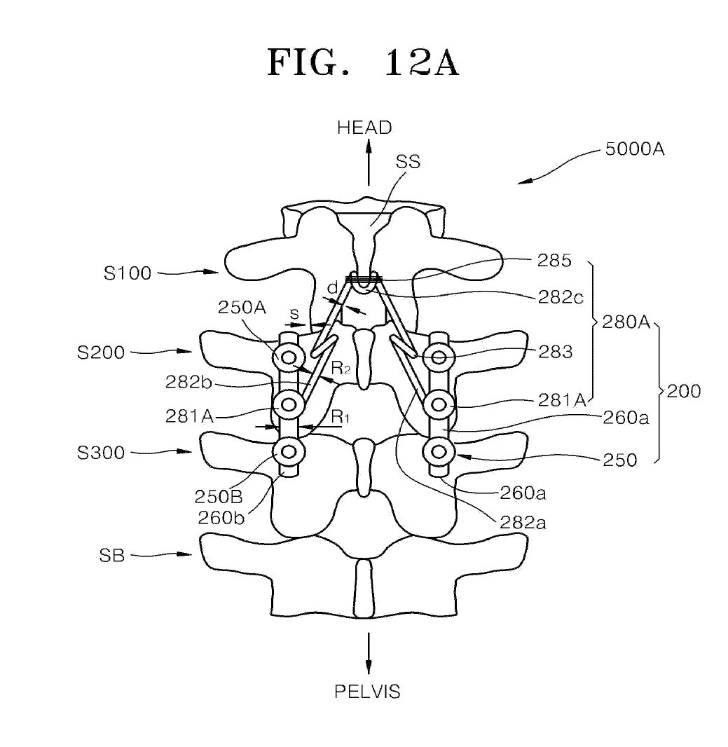

The second rods 282 including the elastic portion 283 are fixed with first the rods 260a and 260b, by using a first fixing portion 281Ba and a second fixing portion 281Bb, respectively. The first fixing portion 281Ba and the second fixing portion 281Bb may comprise a rod clamp 284B2 which surrounds a portion of the first rods 260a and 260b, and a rod mounting portion 284B1 in which a first end 282a or a second end 282b of the second rods 282 are seated. As shown in FIG. 10, a hole 281h is formed in the rod clamp 284B2, a screw groove is formed in an inner sidewall of the hole 281h, the rods 260a and 260b are inserted in the rod clamp 281 B2, an engaging member 284B4, having a helix corresponding to the screw groove of the hole 281h, is inserted in the hole 281h of the rod clamp 284B2 so that the rods 260a and 260b are fixed with the rod clamp 284B2. Similarly, the screw groove is formed in the inside of the rod seating portion 284B1, the first end 282a or the second end 282b of the second rod 282 is mounted in the rod mounting portion 284B1, and then an engaging member 284B4, having the helix corresponding to the screw groove of hole 281h of the rod clamp 284B2, is inserted in the rod mounting portion 284B, so that the second rod 282 can be stabilized with the rod mounting portion 284B1. The rod mounting portion 284B1 may be modified in various shapes in such a way that the rod mounting portion 2811 is formed as one body so that the first rods 260a and 260b and the second rod 282 can be stabilized. A first fixing portion and a second fixing portion according to other embodiments of the present invention will be described later with reference to FIGS. 12A and 12B.

A third end 282c of the second rod 282 including the elastic portion 283 is engaged with the screw-type stabilization member 250 installed to the adjacent spine S100 to stabilize the spine S100 elastically that is adjacent to the stiffly stabilized spines S200 and S300. The elastic portion 283 may be curved portions 201b and 201b′instead of the convolution portion 201a. The first diameter R1 of each of the first rods 260a and 260b may be larger than the second diameter R2 of the elastic portion 283 of the second rod 282. In some embodiments of the present invention, the first diameter R1 may be 5-6 mm so as to connect the engaged screws 250 stiffly, and the second diameter R2 may be 2.5-4.5 mm for elastic stabilization between adjacent spines.

FIG. 10 illustrates the screw 250 as a stabilization member. However, it is obvious to one skilled in the art that the laminar hook-shaped stabilization member 100b shown in FIG. 2B or the pedicle hook-shaped stabilization member 100b may be used as a stabilization member. In addition, as a modified example, it is obvious to one skilled in the art that the third end 282c of the rod 282 is fixed to laminar of the adjacent spine S100 by a hanging stabilization.

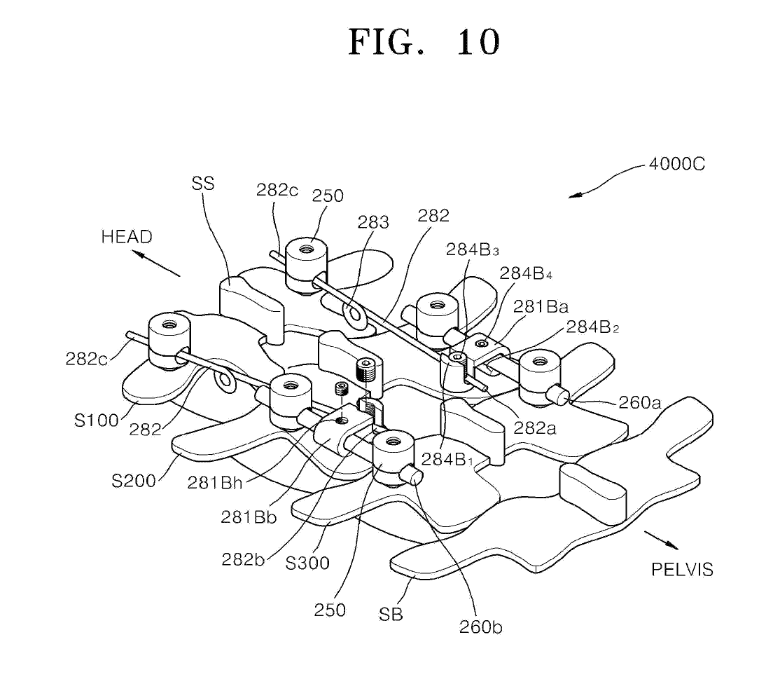

FIG. 11 is a perspective view of a spinal stabilization apparatus 4000D according to another embodiment of the present invention.

Referring to FIG. 11, the spinal stabilization apparatus 4000D comprises laminar hook-shaped stabilization members 100b that are detachably installed to laminars of a second spine S200 and a third spine S300 and symmetrical with respect to spinous processes SS of the second spine S200 and the third spine S300 in a spinal direction. In the some embodiments of the present invention, the rod is a bridge-shaped rod 800c. The bridge-shape rod 800c comprising a pair of elastic portions 801C and a stiffness portion 802c that extends from the elastic portions 801C in the spinal direction to stabilize spines S200 and S300 stiffly.

The stiffness portion 802c of the bridge-shaped rod 800c is engaged with the laminar hook-shaped stabilization member 100b. A third end 803c of the bridge-shaped rod 800c is fixed to the spinous process SS of the first spine S100 by surrounding a portion of the spinous process SS, i.e., a lower portion of the spinous process SS of the first spine S100. However, as also described with reference to FIG. 6A, the third end 803c may also be fixed to the spinous process SS of the first spine S100 by surrounding the upper portion of the spinous process SS of the first spine S100. In addition, the spinal stabilization apparatus 4000D may further comprise a stabilization unit 803, such as a metal thread or a carbon fiber to facilitate the third end 803c and the portion of the spinous process SS of the first spine S100 to be fixed.

The spinal stabilization apparatuses 4000A, 4000B, 4000C, and 4000D illustrated in FIGS. 8 through 11 stiffly stabilize the first and second spines S200 and S300, and the elastic portions 801a, 801b, 282, and 801c stabilize the spines S100 and S200 elastically. The spinal stabilization apparatuses 4000A, 4000B, 4000C, and 4000D according to the present embodiments of the present invention use rods 800a, 800b, 282, and 800c respectively including the elastic portions 801a, 801b, 283, and 801c so that a side effect which can occur from an adjacent fee spine SA as well as the elastically stabilized spines S200 and S300 may be minimized.

In addition, even when side effects occur in adjacent spines of a patient on whom spinal stabilization has been previously performed and re-surgery is conducted, stabilization can be simply achieved by installing a stabilization member and a rod having an elastic portion, according to the present invention without removing the pre-installed stabilization member so that re-surgery can be easily conducted. Since all of the pre-installed stabilization members do not need to be removed during re-surgery in this way, additional internal injury caused by re-surgery may be reduced.

FIGS. 12A and 12B are a plan view and a perspective view of a spinal stabilization apparatus 5000A according to another embodiment of the present invention, respectively.

Referring to FIGS. 12A and 12B, the spinal stabilization apparatus 5000A comprises screws 250 which are installed in at least two adjacent spines S200 and S300, rods 260a and 260b which are engaged with the screws 250, respectively, and connect the screws 250 stiffly in a line, and a bridge-shaped rod 282a which connects elastically a spinous process SS of spine S100 adjacent to the spines S200.

The screw 250 shown in FIG. 2A may be used in the present embodiment as the screw 250 as a stabilization member. The rods 260a and 260b may be engaged with the upper portion of the screw 250. The two rods 260a and 260b connect the screws 250A and 250B stiffly in a line. As shown in FIG. 11, a laminar hook or pedicle hook, instead of the screw 250, may also be used as the stabilization member.

First and second ends 282a and 282b of the bridge-shaped rod 280 are fixed with each of the rods 260a and 260b using a first fixing portion 281a and a second fixing portion 281b, respectively. The first fixing portion 281a and the second fixing portion 281b may comprise a rod mounting portion 2811 which the rods 260a and 260b and the first end 282a and the second end 282b of the bridge-shaped rod 280 pass through and are fixed in by overlapping them. The rod mounting portion 2811 is grooved, for example, laterally to facilitate insertion of the rods 260a and 260b and the first end 282a and the second end 282b of the bridge-shaped rod 280. A screw groove is formed in an inner circumference of a second hole 2812, and an engaging member 2813 having a helix corresponding to the screw groove is joined with the second hole 2812.

The engaging member 2813 is pressurized against the rods 260a and 260b and the first end 282a and the second end 282b of the bridge-shaped rod 280 so that the first fixing portion 281a and the second fixing portion 281b can be engaged with the rods 260a and 260b and the first end 282a and the second end 282b of the bridge-shaped rod 280, respectively. The rod mounting portion 2811 may be formed in various shapes, for example, in such a way that the rod mounting portion 2811 is formed as one body so that the rods 260a and 260b and the first end 282a and the second end 282b of the bridge-shaped rod 280 can be fixed. The first fixing portion 281Ba and the second fixing portion 281Bb shown in FIG. 10 may be used according to other embodiments of the present invention.

A third end 282c of the bridge-shaped rod 280 may be fixed with the spinous process SS by surrounding a lower portion of the spinous process SS of the spine S100 that is adjacent to the stiffly stabilized spines S200 and S300. An elastic portion 283, for stabilizing the spine elastically, is provided between the first end 282a and the third end 282c and between the second end 282b and the third end 282c of the bridge-shaped rod 280. The elastic portion 283 may comprise curved portion 201b and 201b′ instead of the shown convolution portion 201a or together therewith. The first diameter R1 of each of the rods 260a and 260b may be larger than the second diameter R2 of the elastic portion 283. In one embodiments of the present invention, the first diameter R1 may be 5-6 mm so as to connect the screws 250 stiffly, and the second diameter R2 may be 2.5-4.5 mm for elastic stabilization between adjacent spines.

In some embodiments of the present invention, a stabilization unit 285 is used to facilitate the third end 282c to be fixed with the spinous process SS of the first spine S100. An engaging member such as a bolt nut or a wire or a metal thread or a carbon fiber, may be used as the stabilization unit 285. The third end 282c may be shaped into a prop to accommodate the lower portion of the spinous process SS stably, similar as will be illustrated in FIGS. 7A and 7B. Modified examples related to the stabilization unit for fixing the bridge-shaped rod to the adjacent spine will now be described.

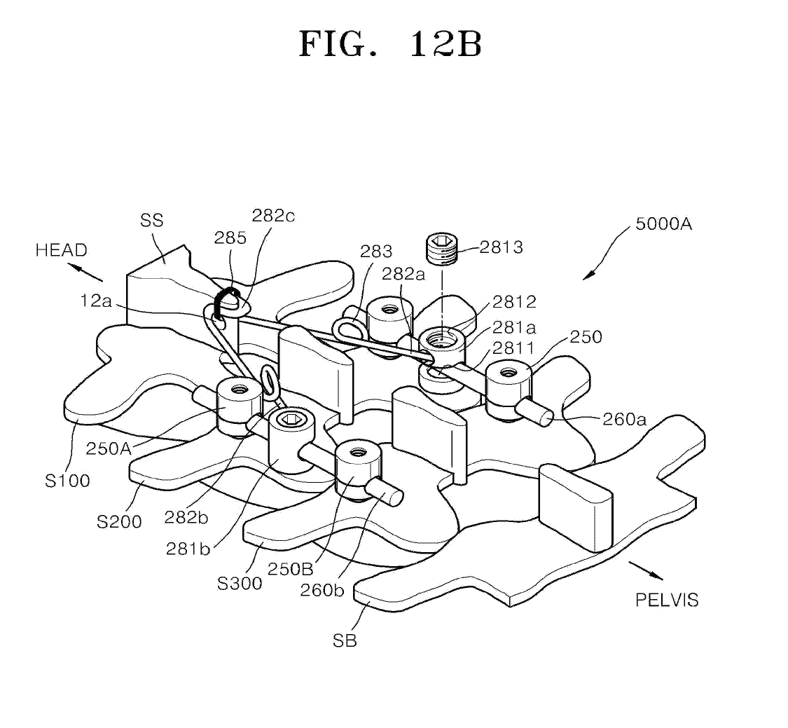

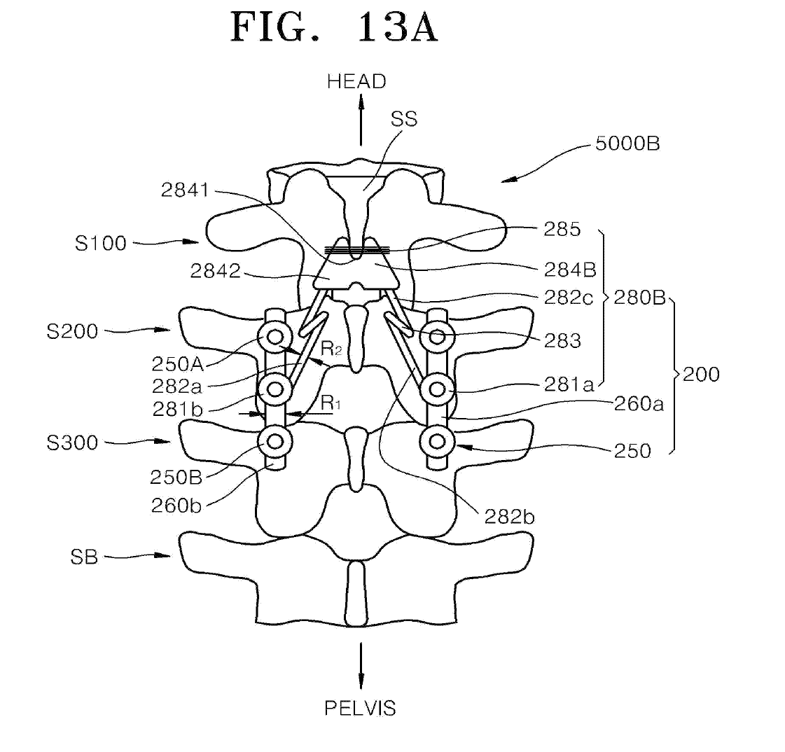

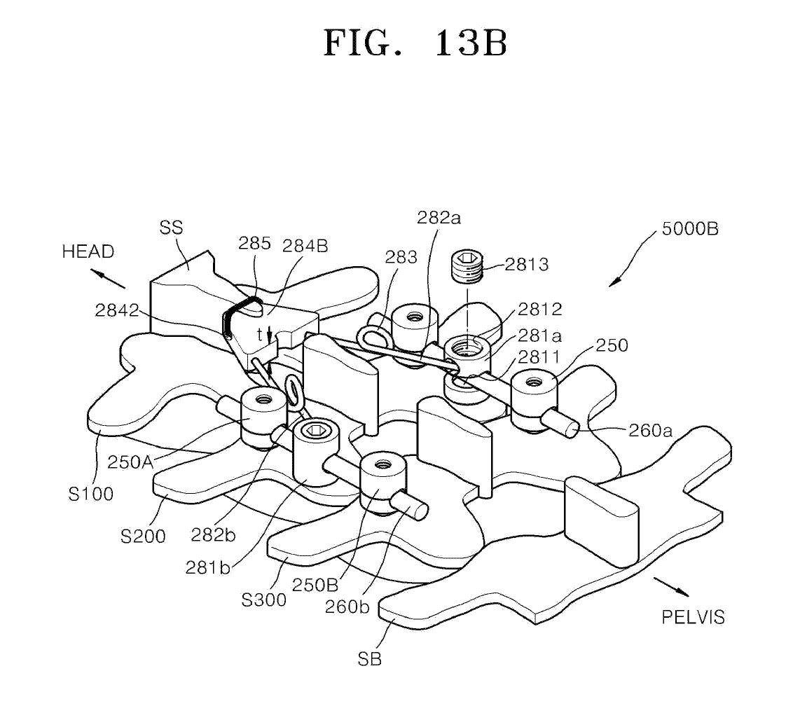

FIGS. 13A and 13B are a plan view and a perspective view of a spinal stabilization apparatus 5000B according to another embodiment of the present invention, respectively.

Referring to FIGS. 13A and 13B, like the spinal stabilization apparatus 5000A described with reference to FIGS. 12A and 12B, a spinal stabilization apparatus 5000B comprises a screw 250 of which a pair is installed to at least two adjacent spines S200 and S300, rods 260a and 260b which are engaged with upper portions of the screw 250, respectively, and connect the screw 250 stiffly in a line, and a bridge-shaped rod 280 which connects a spinous process SS of the adjacent spine S100 to the stiffly stabilized spines S200 and S300 elastically.

Third ends 282c of the bridge-shaped rod 280 may be fixed to a portion of the spinous process SS of the spine S100 using a plate 284B. The plate 284B comprises a concave portion 2841 that correspondingly surrounds at least a portion of the spinous process SS of the adjacent spine S100 and an engaging groove portion 2842 with which the third ends 282c of the bridge-shaped rod 280 are engaged. The plate 284B may have a predetermined thickness t so that the third ends 282c can be inserted therein and engaged therewith. For example, the plate 284B has a thickness t of 4-12 mm that is larger than a second diameter R2 of the rod 280. It is obvious to one skilled in the art that the third ends 282c of the bridge-shaped rod 280 are formed separately as shown in FIG. 12b or are connected to be one body.

The plate 284B may have a sufficient strength to endure a load applied to the spine. The plate 284B may be formed of a stiff material or a flexible material that can be bent uniformly according to movements such as lateral bending, axial rotation, flexion, and extension of the spines S100, S200 and S300. For example, the plate 284B may be formed of a harmless metal such as titanium or a titanium alloy, an elastic material such as a carbon fiber or polymer-based material.

As the stabilization unit 285, for fixing the plate 284B and the spinous process SS together, for example, an engaging member such as a bolt nut, may be used. The engaging member is inserted into a perforated hole of the spinous process SS to engage the plate 284B with the spinous process SS. Alternatively, as the stabilization unit, for facilitating engagement of the plate 284B and the spinous process 12, a harmless metal thread or a wire such as a carbon fiber may be used. For example, a hole is formed in the spinous process SS and the plate 284B and the spinous process SS are engaged together by inserting into the hole and binding the metal thread or wire.

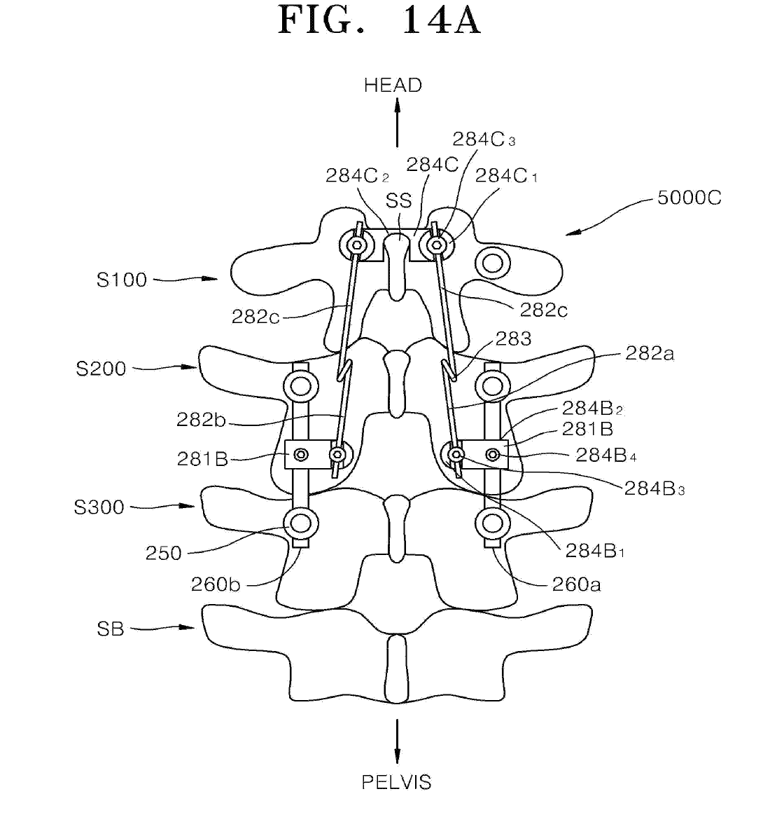

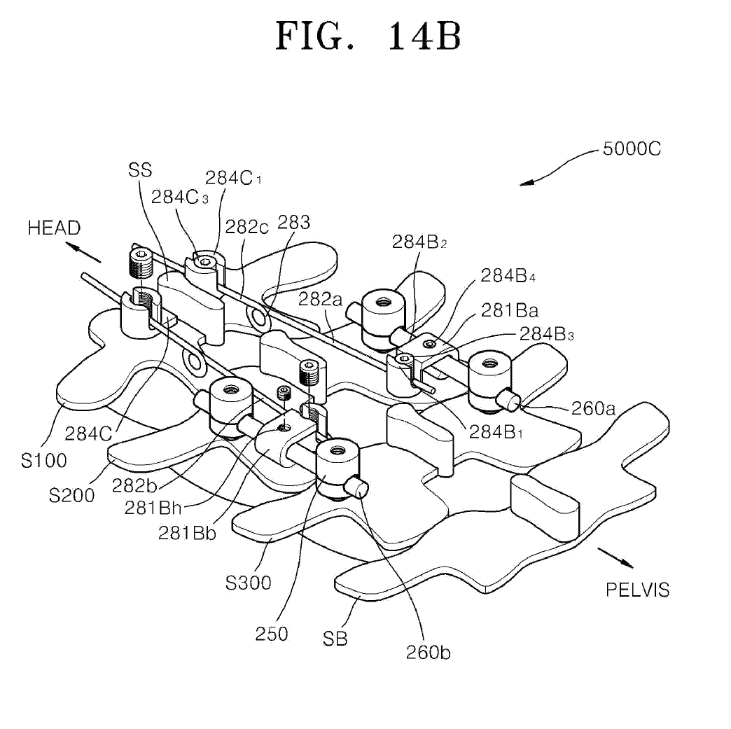

FIGS. 14A and 14B are a plan view and a perspective view of a spinal stabilization apparatus 5000C according to another embodiment of the present invention, respectively.

Referring to FIGS. 14A and 14B, like the spinal stabilization apparatuses 5000A and 5000B described with reference to FIGS. 12A through 13B, the spinal stabilization apparatus 5000C comprises a screw 250 of which a pair is fixed to at least two adjacent spines S200 and S300, rods 260a and 260b which are engaged with upper portions of the screw 250, respectively, and which connect the screws 250 stiffly in a line and each have a first diameter R1, and a bridge-shaped rod 282c which connects spinous processes SS of the spine 100 adjacent to the spines S200 and S300 stiffly-stabilized by the rods 260a and 260b elastically.

The bridge-shaped rod 282C is elastically engaged with the spinous process SS of the adjacent spine S100 using a stabilization unit 284C. The stabilization unit 284C comprises rod mounting portions 284C1 on which a third end 282c of the bridge-shaped rod 282C is mounted and a concave portion 284C2 which correspondingly surrounds the top portion of the spinous process SS. A screw groove is formed in each of the rod mounting portions 284C1 of the stabilization unit 284C, the third end 282c is mounted in the rod mounting portions 284C1 and then the engaging member 284C3 having a helix corresponding to the screw groove is inserted in the screw groove so that the bridge-shaped rod 282C is engaged with the stabilization unit 284C.

A first end 282a and a second end 282b of the bridge-shaped rod 282C are engaged by a first fixing portion 281Ba and a second fixing portion 281Bb, respectively. Other embodiments of the first fixing portion 281Da and the second stabilization portion 281Db will now be described.

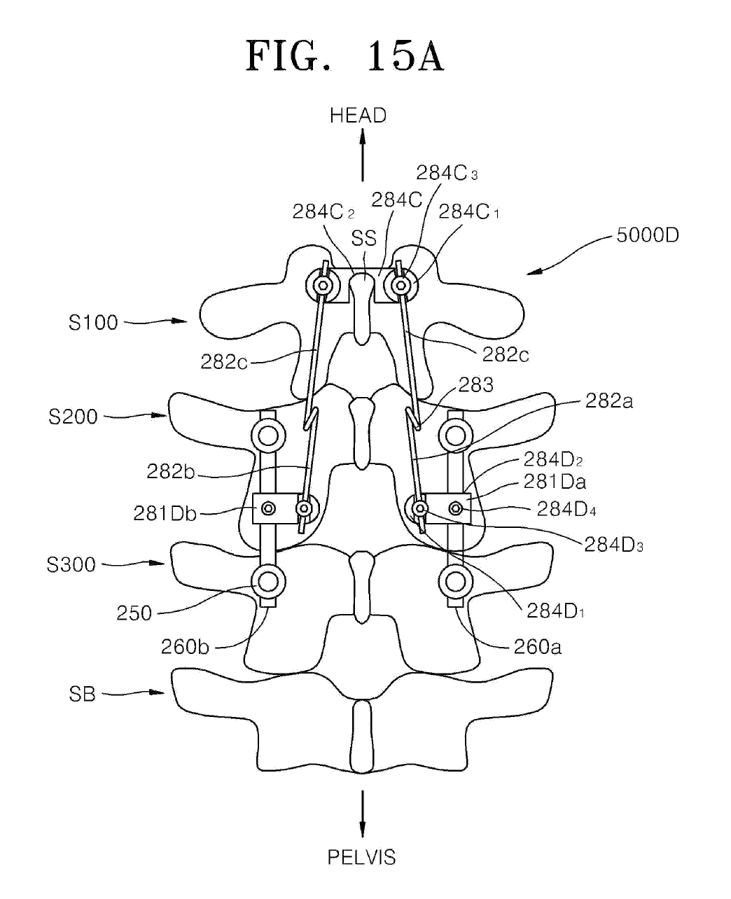

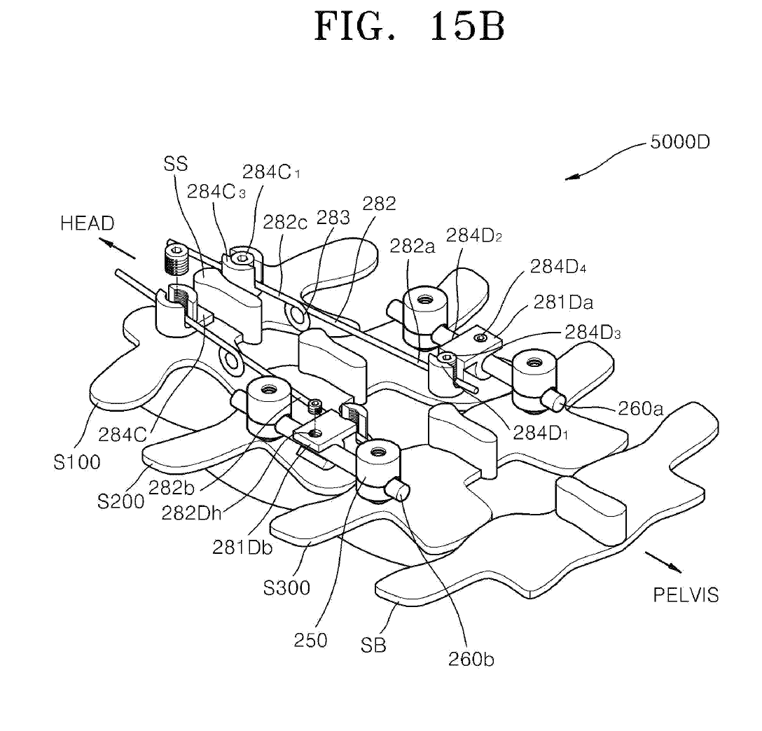

FIGS. 15A and 15B are a top view and a perspective view of a spinal stabilization apparatus 5000D according to another embodiment of the present invention, respectively.

Referring to FIGS. 15A and 15B, a first fixing portion 281Da and a second fixing portion 281Db comprise a rod clamp 284D2 which correspondingly surrounds a portion of rods 260a and 260b and a rod mounting portion 284D1 on which a first end 282a or a second end 282b of a bridge-shaped rod 282c are mounted. As shown in FIGS. 15A and 15B, a hole 282Dh is formed in the first fixing portion 281Da and the second fixing portion 281Db, respectively, a screw groove is formed in an inner sidewall of the hole 282Dh, rods 260a and 260b are inserted into the rod clamp 281Da and then an engaging member 284D4 having a helix corresponding to the screw groove is inserted so that the rod clamp 284D2 and the rods 260a and 260b can be engaged.

Similarly, a screw groove is formed in an inner wall of a groove formed in a rod mounting portion 284D1 of the first fixing portion 281Da and a second fixing portion 281Db, the first end 282a or the second end 282b of the bridge-shaped rod 282c is mounted in the rod mounding portion 284D1, an engaging member 284D3 having a helix corresponding to the screw groove is inserted in the screw groove so that the bridge-shaped rod 282c can be engaged with the spinous process SS of the spine S100.

The first and second fixing portions 281Ba and 281Bb shown in FIGS. 14A and 14B and the first and second stabilization portions 281Da and 281Db shown in FIGS. 15A and 15B can be discriminated in that the rod clamps 284B2 and 284B4 clamp the rods 260a and 260b inward and the rod clamp 284D2 clamps the rods 260a and 260b outward, and thus, a selection of their use may be properly selected as necessary. The first fixing portion 281Ba shown in FIG. 14A and the second fixing portion 281Db shown in FIG. 15B are different from the first and second fixing portions 281a and 281b shown in FIGS. 12A through 13B in that the rods 260a and 260b and the bridge-shaped rod 282 are stabilized independently. A first fixing portion and/or a second fixing portion having various shapes according to embodiments of the present invention may be properly selected according to the size of the spines of a patient and a surgery method.

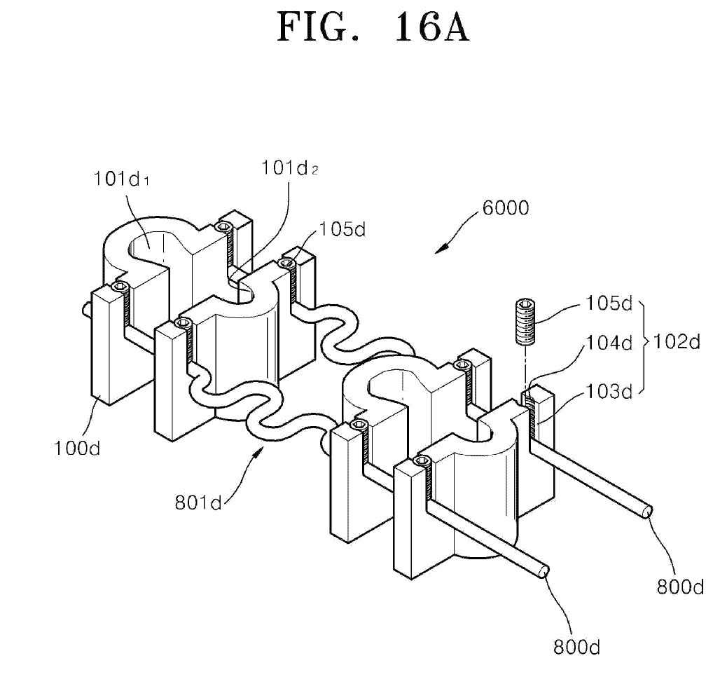

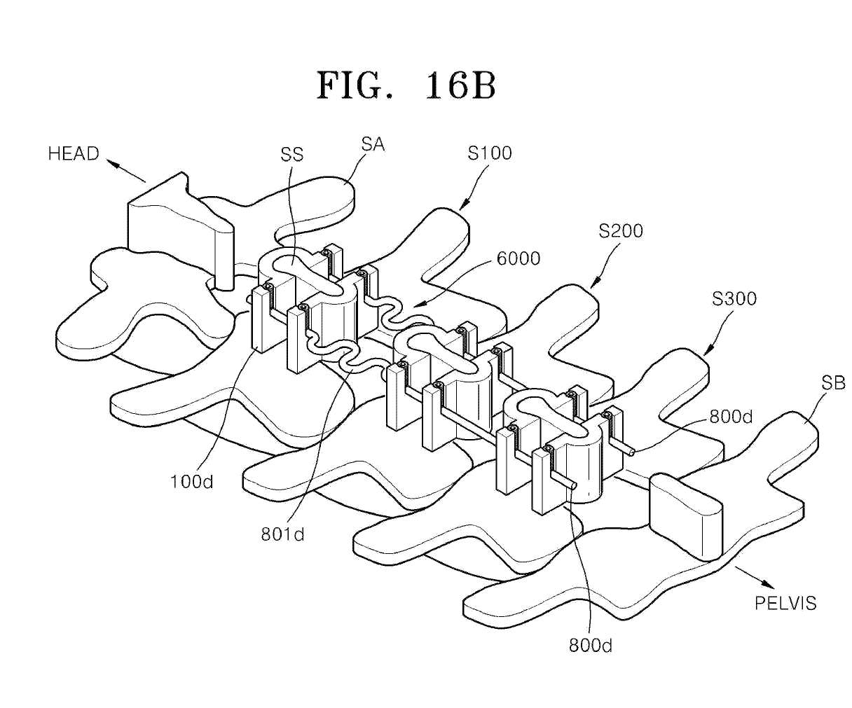

FIGS. 16A and 16B are perspective views of a spinal stabilization apparatus 6000 according to another embodiment of the present invention, respectively.

Referring to FIGS. 16A and 16B, the spinal stabilization apparatus 6000 according to the present embodiment comprises a rod 800d having an elastic portion 801d for stabilizing the first spine S100 and the second spine S200 elastically, as described above. FIGS. 16A and 16B illustrate only a curved portion as the elastic portion 801d, however a convolution portion may be used as the elastic portion. The rod 800d stabilizes the first spine S100 and the second spine S200 elastically as shown in FIG. 16A, however a rod with a convolution portion or a curved portion can be provided between the second spine S200 and the second spine S300 so that the second spine S200 and the third spine S300 can be stabilized elastically. In addition, a stiffness portion which stabilizes the spines S200 and S300 stiffly may be provided, by increasing the diameter of the rod 800d between the second and third spines S200 and S300. A stabilization member 100d, for engaging the rod 800d, comprises a pair of seating portions 101d1 and 101d2 which can correspondingly accommodate a upper portion and lower portion of the spinous process SS, respectively. The pair of seating portions 101d1 and 101d2 are disposed to face each other with each spinous process SS sandwiched by the pair of seating portions 101d1 and 101d2. The pair of seating portions 101d1 and 101d2 are installed by pressurizing the pair of mounting portions 101d1 and 101d2 against the sandwiched spinous processes SS.

The rod 800d is stabilized with a rod mounting portion 102d of the stabilization member 100d. The rod mounting portion 102d comprises a mounting portion 103d in which the rod 800d is mounted, an inner circumference 104d in which a helix is formed, and an engaging member 105d which can screw-engage with the inner circumference 104d. In the drawings, the open type mounting portion 103d, in which the rod 800d are mounted, is shown. However, a perforation type mounting portion, which can be engaged by a method in which the rod 800d can pass through the perforation type mounting portion, may also be included in the spirit of the invention.

The spinal stabilization apparatus 6000 according to the present embodiment uses the rods 800a, 800b, and 800c including the elastic portion 801d so that side effects that may affect the adjacent, free spines SA and SB can be minimized.

While the present invention has been particularly shown and described with reference to exemplary embodiments thereof, it will be understood by those skilled in the art that various changes in form and details may be made therein without departing from the spirit and scope of the invention as defined by the following claims.

1-4. (canceled)

5. A spinal stabilization apparatus comprising:
at least one stabilization member which is installed to a first spine; and
a rod comprising one end engaged with the stabilization member, the other end including a hook portion installed to a second spine adjacent to the first spine, and a convolution portion formed by rolling the rod at least once parallel with an extension direction of the rod from the first spine to the second spine or a curved portion protruding at least once perpendicular to the extension direction.

6. The apparatus of claim 5, wherein the stabilization member comprises at least one screw installed to a pedicle of the first spine.

7. The apparatus of claim 5, wherein the stabilization member comprises at least one laminar hook which is installed to at least one of a superior laminar and an inferior laminar of the first spine.

8. The apparatus of claim 5, wherein the stabilization member comprises at least one pedicle hook which is installed to at least one of a superior pedicle and an inferior pedicle of the first spine.

9. The apparatus of claim 5, wherein the hook portion of the rod is fixed by grabbing a portion of a spinous process of a superior laminar of the second spine or a portion of a superior laminar of the second spine.

10-14. (canceled)

15. A spinal stabilization apparatus comprising:
a plurality of stabilization members installed to at least three adjacent spines; and
a rod engaged with the stabilization member, the rod comprising an elastic portion for stabilizing the two adjacent spines elastically and a stiffness portion extending from one end or both ends of the elastic portion for stabilizing the other spine stiffly.

16. The apparatus of claim 15, wherein the elastic portion is a convolution portion formed by rolling the rod at least once in an extension direction from the one spine to the other spine or a curved portion protruding at least once perpendicular to the extension direction.

17. The apparatus of claim 15, wherein the stabilization members comprise at least one of screw, laminar hook and pedicle hook.

18. The apparatus of claim 15, wherein the elastic portion and the stiffness portion are formed as a single body and the elastic portion has a first diameter and the stiffness portion has a second diameter that is larger than the first diameter.

19. The apparatus of claim 18, wherein the first diameter is 2-5 mm and the second diameter is 4-7 mm.

20. The apparatus of claim 15, wherein the elastic portion and the stiffness portion are separately formed and one end of the elastic portion and one end of the stiffness portion are commonly engaged with one rod mounting portion of the stabilization member.

21. The apparatus of claim 20, wherein the rod mounting portion comprises:
a protrusion between a plurality of grooves on which the end of the elastic portion or the end of stiffness portion are mounted, the protrusion comprising a helix formed thereon; and
an engaging member able to screw-engage with the grooves of the protrusion,
wherein one of the commonly-engaged ends of the elastic portion and the stiffness portion is mounted in the groove of the rod mounting portion, the other thereof includes a disk with a through-hole which is formed to be able to be detachably installed to the protrusion and the disk is pressurized together with the one of the commonly-engaged ends by the engaging member.

22. The apparatus of claim 21, wherein the stiffness portion comprises two subrods and the grooves are formed in a line and each end of the subrods is mounted on each of the grooves.

23. A spinal stabilization apparatus comprising:
a plurality of stabilization members which are symmetrically installed to a pedicle or a laminar of first and second adjacent spines with spinous processes of the first and second adjacent spines between the stabilization members;
a first rod and a second rod which are engaged with the stabilization members to stabilize the first spine and the second spine stiffly; and
a bridge-shaped rod comprising first and second ends engaged with the stabilization members, respectively, a third end correspondingly surrounding a portion of a spinous process of a third spine adjacent to at least one of the first spine and the second spine and installed to the spinous process, and an elastic portion for elastically stabilizing the third spine, the elastic portion disposed between the first end and the third end and between the second end and the third end.

24. The apparatus of claim 23, wherein the elastic portion is a convolution portion formed by rolling the bridge-shaped rod at least once in an extension direction from the first spine to the second spine or a curved portion protruding at least once perpendicular to the extension direction.

25. The apparatus of claim 23, wherein the stabilization members comprise screws, laminar hooks and pedicle hooks.

26. The apparatus of claim 23, wherein the third end comprises a seating portion which accommodates at least one of a upper and an lower portion of the spinous process.

27. The apparatus of claim 23, wherein the bridge-shaped rod comprises a stiffness portion for stabilizing the first spine and the second spine stiffly.

28. A spinal stabilization apparatus comprising:
a rod comprising two ends, a convolution portion formed by rolling the rod at least once in an extension direction of the rod or a curved portion protruding at least once perpendicular to the extension direction between the two end; and
a stabilization member comprising a plurality of a pair of seating portions for accommodating respectively an upper portion and a lower portion of spinous process of at least two adjacent spines, and a rod mounting portion for engaging with the rod.

29. The apparatus of claim 28, wherein the rod mounting portion comprises:
an open type mounting portion or a perforation type mounting portion on which the rod is mounted;
an inner circumference in which a helix is formed; and
an engaging member which is able to screw-engage with the inner circumference to pressurize the rod.
